(19) United States
(12) Patent Application Publication
(10) Pub. No.: US 2026/0248693 A1
(43) Pub. Date: Aug. 27, 2026

(54) Methods, Kits, And Photothermal Compositions For Removing Nail Coatings

(71) Applicant: Liqwd, Inc., Santa Barbara, CA (US)

(72) Inventors: Craig J. Hawker, Santa Barbara, CA (US); Christopher M. Bates, Santa Barbara, CA (US); Javier Read de Alaniz, Santa Barbara, CA (US); Morgan Bates, Santa Barbara, CA (US); Kaitlin Albanese, Santa Barbara, CA (US)

(21) Appl. No.: 19/547,138

(22) Filed: Feb. 23, 2026

Related U.S. Application Data
(60) Provisional application No. 63/762,075, filed on Feb. 23, 2025.
(60) Provisional application No. 63/831,835, filed on Jun. 27, 2025.

(51) Int. Cl. A61K 8/02; A45D 29/00; A45D 29/11; A45D 29/16; A45D 29/18; A61K 8/49; A61Q 3/02; A61Q 3/04

(52) U.S. Cl. CPC A61K 8/025; A45D 29/007; A45D 29/11; A45D 29/16; A45D 29/18; A61K 8/4986; A61Q 3/02; A61Q 3/04; A45D 2200/25; A61K 2800/43; A61K 2800/95

(57) ABSTRACT

Methods, kits fand photothermal compositions for removing nail coatings are described. The nail coatings include a thermally responsive material, such as thermally expandable microspheres, and a nail product to be removed (e.g. nail polish, gel, dip, nail art, and/or artificial nail. Optionally, a bottom coating, such as lipoic acid, is in contact with the nail surface. The method includes (ii) applying a removal stimulus to a removal layer containing a photothermal additive to induce heat generation and cause at least partial expansion, shape change, and/or phase change of the thermally responsive material or partial detachment of the nail product. Optionally the method includes applying the photothermal nail composition on top of a nail coating prior to step (ii).

### CROSS REFERENCE TO RELATED APPLICATION

This application claims priority to U.S. Provisional Patent Applications No. 63/762,075, filed Feb. 23, 2025, and U.S. Provisional Patent Applications No. 63/831,835, filed Jun. 27, 2025, the disclosures of which are incorporated herein by reference.

### FIELD OF THE INVENTION

The disclosed invention is generally in the field of cosmetics, particularly in the field of on-demand removal of nail coatings.

### BACKGROUND OF THE INVENTION

Commercially available nail enhancements, which are formed on nails, may be classified into different categories based on their composition, chemistry, and method of use, for example: (1) nail polishes, also known as lacquers, varnishes, or enamels; (2) gels, including acrylics; (3) dip powder systems; and (4) artificial nails, such as pieces of shaped plastic that can be attached to a nail using gel, dip, glue, or other types of adhesives; as well as combinations thereof. Nail polishes typically include various solid components that are dissolved and/or suspended in non-reactive solvent(s). Upon application and drying, the solids deposit on the nail surface as a clear, translucent, or colored film. Nail polishes are easily scratched and are readily removable with solvent, usually within one minute and if not removed as described, will chip or peel from the natural nail in one to five days. The ability to easily remove nail polishes when desired is a benefit for users, but the lack of mechanical robustness and longevity is a major downside.

In contrast to nail polishes, commercially available gel products applied by a salon or purchased in at-home kits last for up to two weeks without damage. This improved longevity has made gel products popular, and they are available in a variety of styles. The reason gel products are more mechanically robust is related to the different mechanism by which gel manicures solidify compared to conventional nail polishes. Each layer is applied to the nail, for example, by painting it on with a brush, and then cured under light, e.g., an ultraviolet (UV) light or light-emitting diode (LED). The curing process results in solidification of the as-applied liquid layer via a polymerization reaction that results in crosslinking, yielding a coating that is significantly stronger and longer-lasting than conventional nail polishes such as nitrocellulose-based formulations. Procedurally, this process is usually repeated several times per nail. For example, a common process of applying gel products involves: (1) first removing any existing product, optionally with additional nail-surface preparation such as buffing, grinding, or wiping with a solvent, such as isopropanol; (2) applying a base coat; (3) optionally applying one or more color coats to change the appearance of the product; and (4) applying a top coat to create an appealing surface finish. After steps 2, 3, and 4, each as-deposited liquid layer is exposed to light, e.g., a UV lamp and/or LED, to solidify it before moving on to the next one. Sometimes, fingerless gloves are worn to keep UV or LED rays off the hands.

Chemically, gel products are mixtures of monomers, oligomers, and/or polymers, often with additional additives, all of which tune the formulation properties in the liquid state, its reactivity, and the corresponding properties in the solid state after crosslinking. Products such as UV gel nails usually include acrylate, methacrylate, acrylamide, and/or methacrylamide monomers, oligomers, and/or polymers, or other components that undergo a radical-mediated polymerization as initiated by one or more photoinitiators present in the formulation. A related type of product known as acrylic liquid-and-powder nails also involves the solidification of similar monomers, oligomers, and/or polymers by a free-radical polymerization process, but in this case, initiation is induced by thermal initiator(s) such as peroxides present in the powder when it comes in contact with the liquid, rather than a photoinitiator creating radicals under UV or LED light.

Many users enjoy gel products because they last longer than nail polishes. However, this improved longevity makes them more difficult to remove from the nail. If a user desires a different nail enhancement or simply to remove the gel product, often times grinding and/or mild washing and/or heating in a solvent, such as acetone, for an extended period of time (e.g., 10 to 30 minutes) is required. Both of these removal techniques can damage a user's nails, making them undesirable and potentially unhealthy but essentially unavoidable with gel products today.

An even longer-lasting nail product than gels and acrylics gaining popularity is known as dip powder. Dip powder systems involve multiple components in a kit that are applied sequentially. First, the surface of the nail can be prepared as desired by washing, buffing, grinding, dehydrating, or the like. Second, a “base” coat is applied in the liquid (or viscous liquid) state. Third, before the applied base coat fully solidifies, the nail is quickly dipped into a solid powder that sticks as a separate layer to the surface of the base coat. Any excess powder can be lightly brushed off as needed. There are different types of powders that can be selected based on the user's desired appearance for the nail, for example, a wide variety of colors and styles, such as sparkles, are commercially available. This process of applying the base coat followed quickly by dipping the wet surface into a powder can be repeated multiple times, and the same or different powders can be used in each repetition. Various other products can be included in the kit and applied before, during, or after different parts of the process, for example, a separate “activator” solution might be used after the last powder step (or, alternatively, after each powder step) to promote more complete and/or faster solidification of the base layer(s). Often the last step of the process involves applying a “top” coat to create an appealing visual look and tactile sensation. Buffing may also be used to smooth the surface at various points in the overall application process.

Dip powder systems use a different type of chemistry than the aforementioned nail polishes and gel/acrylic products. Although the base and top coat layers of a dip powder system may contain multiple components, such as additives like poly(methyl methacrylate), the key ingredients in each are cyanoacrylate-based monomers, oligomers, and/or polymers. Cyanoacrylates can be applied to the nail or dip-powder-coated layers as a liquid, but the as-applied liquid spontaneously solidifies within minutes without any additional stimulus because the small amount of water present everywhere (e.g., in air, on the nail, in any underlying layers) initiates anionic polymerization. (The aforementioned “activator” solution is an accelerator such as a tertiary aryl amine that also promotes anionic polymerization.) This chemistry is analogous to super glue, and like super glue, these coatings are mechanically robust, strong, and long-lasting. Dip powder systems are therefore even more difficult to remove than gel products. Removal often involves aggressive mechanical grinding and abrasion plus swelling with solvent for extended periods of time, which as mentioned above, can damage a user's nails. Such damage may limit the frequency at which users can change dip powder nail products or even cause a temporary pause in the use of nail-enhancement products to provide time for the nail to heal and regenerate after removing one product but before applying another.

Artificial nails often use gel (e.g., crosslinkable acrylates) or dip (cyanoacrylate) chemistry to glue a plastic piece to a user's nail. Therefore, they suffer from the same difficulty in removal as described above for gel and dip powder systems.

Therefore, while gels, dip powder systems, and artificial nail enhancements are popular options for achieving long-lasting and aesthetically pleasing manicures and pedicures, their removal remains a significant challenge with the potential to damage the nail bed and cause discomfort, bleeding, infection, nail loss, and/or prolonged health issues.

Thus, there remains a need for providing method for removal of nail coatings that address and overcome the challenges associated with removing such commercially available nail coatings.

Accordingly, it is an object of the invention to provide nail compositions that enable nail coatings to be removed with ease when removal is desired.

It is a further object of the invention to provide improved methods for removing nail coatings.

### BRIEF SUMMARY OF THE INVENTION

Described herein are photothermal nail compositions which allow for facile removal of nail products and methods for removing such nail coatings from a subject's nail(s).

In one non-limiting instance, a photothermal nail composition includes: at least one photothermal additive; and a cosmetically acceptable carrier and/or a matrix. The photothermal additive can be a carbon material, an organic dye, an inorganic compound, an inorganic element, or an inorganic alloy, or a combination thereof. The photothermal additive is generally present at a concentration ranging from about 0.001 wt. % to 99 wt. % of the total weight of the photothermal nail composition.

Various methods are described herein that can be used in various applications in the nail care industry. The nail product(s) present in nail coatings are at least partially removable by exposure to a suitable removal stimulus, as described in the exemplary removal methods detailed below.

The nail coating includes a thermally responsive material (such as a shape changing, phase changing and/or thermally expandable material) and a nail product. Optionally, the nail coating is on top of a bottom coating that directly contacts the subject's nail surface. In some instances the thermally responsive material is included in one or more layers, where at least the first layer containing the thermally responsive material is in contact with the surface of the subject's nail or is in contact with the bottom layer, when such a layer is present.

Optionally, the nail coating includes a plurality of layers, where the top layer is or includes the nail product. Further, at least one layer in the plurality of layers in the nail coating includes the thermally responsive material.

In one non-limiting instance, a method of removing a nail coating, such as described above, from a subject's nail includes the steps of:
(i) applying a photothermal nail composition, as described herein, on top of a nail coating; and
(ii) applying a removal stimulus to the removal layer for a sufficient amount of time to induce heat generation by the photothermal additive(s).

After step (i), the photothermal nail composition is or forms a removal layer on top of the nail product.

In some instances, the amount of heat generated during step (ii) is sufficient to cause at least partial debonding, delamination, and/or detachment of the nail product from the subject's nail or from an interface with the layer(s) containing the thermally responsive material. In some instances, the amount of heat generated during step (ii) is sufficient to cause at least partial expansion, shape change, and/or phase change of the thermally responsive material.

In another non-limiting instance, such as when at least one photothermal additive is present on top of the nail coating or is included in the nail coating, a method of removing the nail coating, such as described above, from a subject's nail includes the steps of:
(i′) applying a removal stimulus to the nail coating for a sufficient amount of time to induce heat generation by the photothermal additive(s) and cause at least partial debonding, delamination, or detachment of a nail product from the subject's nail or from an interface with at least one layer in the nail coating that contains thermally responsive material; or
applying a removal stimulus to the nail coating for a sufficient amount of time to induce heat generation by the photothermal additive(s) and cause at least partial expansion, shape change, and/or phase change of the thermally responsive material in the nail coating.

At least one layer of the plurality of layers in the nail coating includes a thermally responsive material, such as a shape changing, phase changing and/or thermally expandable material. In some cases, at least two layers of the plurality include such a thermally responsive material. It is understood that the heat generated upon exposure of the photothermal additive to the removal stimulus is sufficient to activate (e.g., expand) or at least partially activate the thermally responsive material in at least one portion of at least one layer of the plurality that includes the thermally responsive material. Such expansion, shape change, and/or phase change of the thermally responsive material typically causes lifting, peeling, debonding, and/or cracking of the nail product.

In some instances, the methods also include a step of scoring the surface of the top layer of the nail coating prior to step (ii) or (i′) to promote or define the cracking pattern upon expansion, shape change, and/or phase change, and facilitate removal of the nail product.

In some instances, the top layer of the layer(s) containing the thermally responsive material is applied as a single layer. The single layer can have a thickness that is similar to the thickness of a builder gel, where a builder gel is a “hard” gel that is typically more viscous and is applied thicker than a “soft” gel to impart structure on the nail. Hard gels are not designed to be removed with solvent which is why they can be thicker and more crosslinked. By contrast, soft gels are more open/porous and can be removed with solvent, and are typically thinner.

The removal stimulus can be applied from any suitable source. In some instances, the removal stimulus is produced by an electronic device including one or more light sources, such as light-emitting diodes (LEDs), infrared emitters, intense pulsed light (IPL), an arc lamp, or a laser which produce the removal stimulus. In other instances, the light source is an intense pulsed light source, such as that produced by a xenon arc lamp.

Kits useful for practicing the methods described above, and for applying photothermal nail compositions used therein, are also disclosed. For example, kits are provided for nail care applications, such as removing nail coatings and applying photothermal nail compositions for removal of such nail coatings on-demand, by way of application of a suitable removal stimulus.

### BRIEF DESCRIPTION OF THE DRAWINGS

The accompanying drawings illustrate several embodiments of the disclosed compositions and methods thereof which together with the description, serve to explain the principles of the disclosed methods and compositions.

FIG. 1A shows a non-limiting method (parts A, B, C, and D) for removal from the surface of a fingernail a multilayer nail coating 1000, containing a nail product 1100 in the top layer followed by a second layer of thermally responsive material 1200b, and a first layer of thermally responsive material 1200 a (see FIG. 1A, part A). As depicted in part B, a photothermal layer 2000 is applied to the surface of the top layer 1100 containing the nail product. In part C, a removal stimulus 3000 is applied, which causes heat generation by the photothermal layer 2000, which is conducted to the underlying nail coating 1000 and causes the thermally responsive material to change shape, phase change, and/or expand in at least a portion of the second layer of thermally responsive material 1200b to form 1200b′, thereby facilitating removal of the nail coating from the fingernail (as depicted in part D).

FIG. 1B shows a non-limiting method (parts A, B, C, and D) for removal from the surface of a fingernail a multilayer nail coating 1000′ containing a nail product 1100 in the top layer followed by a single layer containing a thermally responsive material 1200 (see FIG. 1B, part A). As depicted in part B, a photothermal layer 2000 is applied to the surface of the top layer 1100 containing the nail product. In part C, a removal stimulus 3000 is applied, which causes heat generation by the photothermal layer 2000, which is conducted to the underlying nail coating 1000′ and causes the thermally responsive material to change shape, phase change, and/or expand in at least a portion of a thermally responsive layer, thereby facilitating removal of the nail coating from the fingernail (as depicted in part D).

FIG. 2 shows a non-limiting method (parts A, B, C, and D) for removal of a multilayer nail coating 1000, that is present over a bottom coating 4000 directly contacting the fingernail. The process depicted in FIG. 2 is the same as the process depicted in FIG. 1A, however with the addition of the presence of a bottom coating 4000 between the fingernail surface and the first layer of thermally responsive material 1200a in the multilayer coating 1000. Further at the end of the process, the bottom coating 4000 remains on the surface of the fingernail, as depicted in part D.

### DETAILED DESCRIPTION OF THE INVENTION

Photothermal nail compositions which allow for facile removal of nail products and methods for removing such nail coatings are described herein.

### I. Definitions

The term “cosmetically acceptable,” as used herein, refers to compositions, formulations, or components thereof that are suitable for use in contact with human keratinous tissue (such as nail or skin) without undue toxicity, incompatibility, instability, allergic response, and the like.

The term “coating” refers to a deposit formed directly or indirectly on at least a portion of a surface of a keratin material, such as a nail surface, where the deposit either adheres to the keratin material or adheres to another coating or material that is in contact with the surface of the keratin material, such as a nail surface (i.e., indirectly deposited on the surface of a keratin material, such as a nail surface). A coating may itself include multiple layers of the same or different materials.

The term “removable” refers to the ability of a nail enhancement (such as formed from one or more nail products) to be removed, detached, or delaminated completely or partially from a nail surface or delaminated completely or partially from an at least partially expanded thermally responsive material, such as a shape changing, phase changing and/or thermally expandable material, at the interface onto which the nail enhancement (i.e. nail product) has been formed. This is performed while causing no damage or minimal damage to the natural nail surface on which the nail enhancement is present. For example, a nail enhancement is considered to be removable when during the removal process the nail enhancement is removed from the nail surface but its removal does not cause tearing of the nail, chipping of the nail, appreciable damage to the nail bed, or removal of significant portions or areas of the nail. A nail enhancement or product is considered to be removable when minor amounts of residue of the multilayer nail coating, or the optional bottom coating, or an adhesive remains after removal and/or minor discoloration of the nail surface results following removal. For example, white spots on the surface of the nail, or small bumps/ridges that can be remedied with a separate application of a nail strengthener, as directed, or buffing, following removal of a removable nail enhancement.

As used herein, the term “nail enhancement” or “nail product” refers broadly to any material that is applied, or product that forms after it is applied, to a natural nail or artificial nail for the purpose of modifying its appearance, structure, strength, durability, or function.

This includes, but is not limited to, nail polish, nail gels, dip powders, acrylics, builder gels, artificial nails, press-on nails, hybrid systems, or other products that serve a similar decorative or structural function for nails. In addition, “nail art” can be applied to such nail enhancements or nail products, and refers to decorative or ornamental elements applied to an artificial nail surface, including, without particular limitation, artificial gemstones, pearls, studs, beads, charms, decals, foils, glitter, and other types of embellishments known in the field of nail care.

“Photothermal” refers to the conversion of light energy into thermal/heat energy when a material, such as a photothermal additive, absorbs light leading to an increase in temperature. As used herein, “photothermal” can also include processes where incident light either directly generates heat through absorption or indirectly generates heat by initiating a photochemical, chemical or physical reaction whose products or intermediates release thermal energy.

The term “volatile” refers to solvent(s) which are capable of evaporating on contact with the nail surface in less than one hour, at room temperature and atmospheric pressure.

The term “effective period of time” or “effective amount of time” refers to the amount of time which is sufficient to cause a desired condition, effect, or outcome to occur. It is typically the minimum span of time required to effectively produce the intended result.

Numerical ranges disclosed herein disclose individually each possible number in such range, as well as any sub-ranges and combinations of sub-ranges encompassed therein. For example, a concentration range or weight percent range, such as from 1% to 2% by weight of the formulation discloses, the individual values and fractions thereof, such as 1%, 1.1%, 1.2%, 1.32%, 1.48% etc., as well as sub-ranges encompassed within.

Use of the term “about” is intended to describe values either above or below the stated value, which the term “about” modifies, in a range of approx. +/−10%; in other instances the values may range in value either above or below the stated value in a range of approx. +/−5%. When the term “about” is used before a range of numbers (i.e., about 1-5) or before a series of numbers (i.e., about 1, 2, 3, 4, etc.) it is intended to modify both ends of the range of numbers or each of the numbers in the series, unless specified otherwise.

### II. Photothermal Nail Compositions

Described herein are photothermal nail compositions that can be used in methods that allow for facile removal of nail product(s) present on a subject's nail when exposed to a suitable removal stimulus. Following application, the photothermal nail composition is or forms a removal layer on top of the nail product.

In one non-limiting instance, a photothermal nail composition includes at least one photothermal additive and a cosmetically acceptable carrier and/or a matrix. The photothermal additive(s) can be carbon material, an organic dye, an inorganic compound, an inorganic element, or an inorganic alloy, or any combination thereof. Generally, the photothermal additive(s) is present at a concentration ranging from about 0.001 wt. % to 99 wt. % of the total weight of the photothermal nail composition.

Generally, when exposed to a suitable removal stimulus, the photothermal additive(s) generate sufficient heat upon exposure to a removal stimulus to produce an increase in temperature in the photothermal nail composition of at least about 5° C., 10° C., 15° C., 20° C., 25° C., 30° C., 35° C., 40° C., 45° C., 50° C., 60° C., 70° C., 80° C., 90° C., 100° C., 110° C., 120° C., 130° C., 140° C., 150° C., 160° C., 170° C., or 180° C., (or an increase in a range of about 5° C. to 180° C., 25° C. to 180° C., 50° C. to 180° C., 75° C. to 180° C., 100° C. to 180° C., 125° C. to 180° C., 150° C. to 180° C., 5° C. to 160° C., 25° C. to 160° C., 50° C. to 160° C., 75° C. to 160° C., 100° C. to 160° C., 125° C. to 160° C., 150° C. to 160° C., 5° C. to 170° C., 25° C. to 170° C., 50° C. to 170° C., 75° C. to 170° C., 100° C. to 170° C., 125° C. to 170° C., or 150° C. to 170° C.) following at least about 30 seconds to about 5 minutes, about 30 seconds to about 4 minutes, about 30 seconds to about 3 minutes, about 30 seconds to about 2 minutes, or about 30 seconds to about 1 minute of exposure to the removal stimulus, as compared to the temperature of the photothermal nail composition prior to exposure to the removal stimulus. Individual values or sub-ranges contained within any of the aforementioned temperature and time ranges are also possible.

In some instances, the concentration of the at least one photothermal additive present is sufficient to generate heat upon exposure to a removal stimulus to produce an increase in temperature in the photothermal nail composition of at least about 5° C., 10° C., 15° C., 20° C., 25° C., 30° C., 35° C., 40° C., 45° C., 50° C., 60° C., 70° C., 80° C., 90° C., 100° C., 110° C., 120° C., 130° C., 140° C., 150° C., 160° C., 170° C., or 180° C., (or an increase in a range of about 5° C. to 180° C., 25° C. to 180° C., 50° C. to 180° C., 75° C. to 180° C., 100° C. to 180° C., 125° C. to 180° C., 150° C. to 180° C., 5° C. to 160° C., 25° C. to 160° C., 50° C. to 160° C., 75° C. to 160° C., 100° C. to 160° C., 125° C. to 160° C., 150° C. to 160° C., 5° C. to 170° C., 25° C. to 170° C., 50° C. to 170° C., 75° C. to 170° C., 100° C. to 170° C., 125° C. to 170° C., or 150° C. to 170° C.) following at least about 30 seconds to about 5 minutes, about 30 seconds to about 4 minutes, about 30 seconds to about 3 minutes, about 30 seconds to about 2 minutes, or about 30 seconds to about 1 minute of exposure to the removal stimulus, as compared to the temperature of the composition prior to exposure to the removal stimulus. Individual values or sub-ranges contained within any of the aforementioned temperature and time ranges are also possible.

When a suitable removal stimulus is applied to the photothermal nail composition for a period of time ranging from about 30 seconds to about 5 minutes, about 30 seconds to about 4 minutes, about 30 seconds to about 3 minutes, about 30 seconds to about 2 minutes, or about 30 seconds to about 1 minute of exposure to the removal stimulus, it is effective to cause the at least one photothermal additive to generate sufficient heat to produce an increase in temperature in the composition of at least about 5° C., 10° C., 15° C., 20° C., 25° C., 30° C., 35° C., 40° C., 45° C., 50° C., 60° C., 70° C., 80° C., 90° C., 100° C., 110° C., 120° C., 130° C., 140° C., 150° C., 160° C., 170° C., or 180° C., (or an increase in a range of about 5° C. to 180° C., 25° C. to 180° C., 50° C. to 180° C., 75° C. to 180° C., 100° C. to 180° C., 125° C. to 180° C., 150° C. to 180° C., 5° C. to 160° C., 25° C. to 160° C., 50° C. to 160° C., 75° C. to 160° C., 100° C. to 160° C., 125° C. to 160° C., 150° C. to 160° C., 5° C. to 170° C., 25° C. to 170° C., 50° C. to 170° C., 75° C. to 170° C., 100° C. to 170° C., 125° C. to 170° C., or 150° C. to 170° C.), as compared to the composition prior to exposure to the removal stimulus. Individual values or sub-ranges contained within any of the aforementioned temperature and time ranges are also possible.

In some instances, the concentration of the photothermal additive ranges from about 0.01 wt. % to 99 wt. %, 0.1 wt. % to 95 wt. %, 0.1 wt. % to 90 wt. %, 0.1 wt. % to 80 wt. %, 0.1 wt. % to 70 wt. %, 0.1 wt. % to 60 wt. %, 0.1 wt. % to 50 wt. %, 0.1 wt. % to 40 wt. %, 0.2 wt. % to 30 wt. %, 0.5 wt. % to 20 wt. %, 1 wt. % to 10 wt. %, 2 wt. % to 5 wt. %, 0.1 wt. % to 3 wt. %, 0.1 wt. % to 2 wt. %, 0.1 wt. % to 1 wt. %, 0.5 wt. % to 5 wt. %, 0.5 wt. % to 4 wt. %, 0.5 wt. % to 3 wt. %, 0.5 wt. % t 2 wt. %, 1 wt. % to 5 wt. %, 1 wt. % to 4 wt. %, 1 wt. % to 3 wt. %, or 2 wt. % to 4 wt. % of the total weight of the photothermal nail composition, as well as individual values or sub-ranges contained within any of the aforementioned ranges.

In some other instances, the cosmetically acceptable carrier of the photothermal nail composition is not present in the resulting removal layer. For example, the carrier can evaporate following application to the top surface of the nail coating, leaving a photothermal coating where 100% or essentially 100% of the removal layer contains the photothermal additive(s).

In some instances, the photothermal nail composition is a liquid and includes a liquid cosmetically acceptable carrier and/or matrix, where the cosmetically acceptable carrier can be evaporated and components of the matrix form a coating therefrom, when the composition is applied to a surface, such as a nail coating. In such instances, the photothermal nail composition is understood to form a photothermal coating, i.e. a removal layer, which is located on top of the nail coating and/or nail enhancement on the surface of a nail.

a. Composition Forms The photothermal nail compositions can have any form suitable for application onto a surface, such as a nail coating. In some instances, the photothermal nail composition is in the form of a sticker and the matrix is or includes an adhesive which can adhere onto the surface of a nail coating. When in the form of a sticker, the photothermal agent/additive is contained within or forms part of the sticker, which is attached/adhered to the top of a nail coating. In sticker form, the composition may be provided on a removable/peelable backing or liner. In some instances, the sticker may be a commercially available sticker, which includes a photothermal additive, such as a carbon material (i.e., carbon black), that generates heat when exposed to an appropriate stimulus, as detailed below. The sticker may have a nail shape or can be cut into a suitable shape for application onto a nail coating.

In some other instances, the photothermal nail composition may be provided as a liquid or as a soft solid, which is applied by a brush or is filled into a marker pen reservoir allowing for the photothermal nail composition to be applied or painted onto a surface, such as a nail coating. When in soft solid form, the photothermal nail composition may have a yield stress such that it can be painted onto the nail and form a solid coating that holds its shape and remains largely on the nail surface without flowing onto the surrounding skin/cuticle area.

In some instances, the thickness of the photothermal nail composition following evaporation of the cosmetically acceptable carrier(s) forms a coating that is about 1 micrometer to 1 mm, 10 micrometers to 500 micrometers, 20 micrometers to 400 micrometers, 30 micrometers to 350 micrometers, 40 micrometers to 300 micrometers, or 50 micrometers to 250 micrometers. In some instances, the coating is uniform in thickness after evaporation of the solvent. In other instances, the coating is not uniform in thickness after evaporation of the solvent, for example, when a photothermal nail composition is used, which may leave thicker streaks after solvent evaporation. In some instances the photothermal nail composition conformally coats the nail enhancement, even if the nail enhancement itself does not have a uniform thickness (i.e., has an irregular surface topography). In some instances, the thickness of the photothermal nail composition in the form of a sticker is 1 micrometer to 5 mm, 10 micrometers to 4 mm, 20 micrometers to 3 mm, 20 micrometers to 2 mm, or 50 micrometers to 1 mm. In some instances, where the photothermal nail composition is provided in the form of a sticker, the sticker has a thickness in the range of about 100 microns to 300 microns, 100 microns to 200 microns, or 100 microns to 150 microns. Individual values or sub-ranges contained within any of the aforementioned thickness ranges are also possible.

In some instances, the photothermal nail composition contains a commercially available nail polish or gel therein, such as by forming a mixture thereof. In some other instances, the photothermal nail composition is not a commercially available nail polish or gel.

### A. Exemplary Photothermal Additives

In some instances, the photothermal additive is or includes an organic dye with a maximum absorption in the range of 400 nm to 1500 nm when considering absorbance across the range of 350 nm to 2000 nm. Examples of suitable organic dyes include, but are not limited to, Epolin Epolight® dyes (such as Epolight® 1125, Epolight® 3801, Epolight®5588), donor-acceptor Stenhouse adducts, azobenzene derivatives, perylene diimide polymer dyes, and combinations thereof.

In some other instances, the photothermal additive is a carbon material such as carbon black, graphene, graphene oxide, reduced graphene oxide, single-walled carbon nanotubes, multi-walled carbon nanotubes, acetylene black, graphite, activated carbon, or carbon nanofibers, or a combination thereof. In some instances, the photothermal additive is or includes carbon nanotubes, such as single-walled carbon nanotubes and/or multi-walled carbon nanotubes.

In still other instances, the photothermal additive is an inorganic compound, an inorganic element, an inorganic alloy, such as indium-tin oxide (ITO), mica, titanium nitride, iron oxide, or plasmonic particle (such as gold nanoparticles, gold nanorods, silver sulfide quantum dots, and silver nanoparticles), or a combination thereof.

Combinations of various types of photothermal additives, such as those described herein, are possible. Other types of known photothermal additives may be used.

In some instances, the photothermal nail composition excludes the presence of any dyes and/or pigments that are known to be used in the field of nail care and used to provide a color to nail polishes or gels. In some instances, the photothermal nail composition contains dyes and/or pigments that are known to be used in the field of nail care and used to provide a color to nail polishes or gels in a concentration of about 0.1 wt. % to 99 wt. % of the total weight of the photothermal composition, as well as individual values or sub-ranges contained within any of the aforementioned ranges.

### B. Cosmetically Acceptable Carrier and Matrices

In some instances, the photothermal nail composition, when in liquid form, includes a suitable cosmetically acceptable carrier and/or matrix which allows for formation of a coating when applied onto a surface, such as a nail coating. Formation of the coating can occur by various manners including, but not limited to evaporation of the carrier(s) that produce a coating or film from component(s) of the matrix. In other instances, formation of the coating can occur without any cosmetically acceptable carrier by applying the liquid matrix formulation followed by curing (solidification) of the matrix using heat and/or light and/or exposure to a chemical curing agent. Without limitation, components of the matrix can be selected from a lacquer, monomers, film-forming polymers, and combinations thereof.

In some instances, the monomers are selected from acrylates, methacrylates, acrylamides, cyanoacrylates, styrenes, epoxies, dithianes, and/or dithiolanes. It is understood that the monomers will polymerize following application to the nail coating, evaporation of the carrier(s) of the composition, and curing, which leads to formation of a coating that forms a removal layer.

In some instances, the film-forming polymers are selected from nitrocellulose, cellulose acetate, cellulose acetate butyrate, ethyl cellulose, hydroxypropyl cellulose, other substituted cellulose derivatives, acrylates copolymer, methacrylates copolymer, dimethicone, substituted dimethicones, polyvinyl butyral, polyurethane resins, tosylamide/formaldehyde resin, and the like. It is understood that the film-forming polymer(s) form a coating upon application to the nail coating and optional evaporation of carrier(s) of the composition, and the resulting coating is the removal layer.

In some instances, the cosmetically acceptable carrier is a solvent, such as an organic solvent or water. Various suitable solvents/carriers can be used to help the spreadability of photothermal nail compositions and/or keep the ingredients dissolved in the compositions during application, but which evaporate after the composition has been applied to a surface to induce formation of a coating. Non-limiting examples of suitable solvents and carriers, include water, acetone; esters such as ethyl acetate, propyl acetate, and butyl acetate; linear and branched alcohols, such as ethanol, propanol, isopropanol, hexanol, and the like;

aromatic or cyclic alcohols, such as benzyl alcohol, cyclohexanol, and the like; saturated C₁₂ to C₃₀ fatty alcohols, such as lauryl alcohol, myristyl alcohol, cetyl alcohol, stearyl alcohol, behenyl alcohol, and the like. In some instances, non-aqueous or hydrophobic auxiliary solvents can be used to prepare compositions that are substantially water-free products, such as nail lacquers. Such solvents/carriers have a suitable volatility level such that they evaporate quickly, such as within 5 minutes, 4 minutes, 3 minutes, 2 minutes, 1 minute, 45 seconds, 30 seconds, 15 seconds, 10 seconds, or less following application of the photothermal nail composition to a surface.

In some instances, the total concentration of all of the solvents/carriers in the photothermal nail composition is at least 0.5% and less than about 99%, 90%, 80%, 70%, 60%, 50%, 40%, 30%, 20%, 10%, 5%, or 1% by weight relative to the total weight of photothermal nail composition. In some instances, the total concentration of all of the solvents/carriers in the photothermal nail composition ranges from about 1% to 99% by weight relative to the total weight of photothermal nail composition, as well as individual values or sub-ranges contained within. Total concentration refers to the concentration of all of the solvents/carriers, photothermal additive(s), and any other components of the matrix that are present in the photothermal nail composition before the evaporation of any volatile solvent/carrier therefrom.

In some other instances, the photothermal nail composition is a solid and does not include any liquid carrier/solvent and a solid matrix is present, such as when the composition is in the form of a sticker. In such sticker forms, the photothermal additive(s) are held within a solid matrix forming a layer, which may be adhesive or may have an additional adhesive layer present thereon. In some instances, the sticker and layers thereof are provided on a backing or liner, such as paper, which is peelable/removable. Thus, the sticker may be removed from the backing or liner prior to or during application of the sticker onto a nail surface or onto any nail products/enhancements on a nail surface.

### C. Optional Cosmetically Acceptable Excipients

In some instances, the photothermal nail composition further includes at least one cosmetically acceptable excipient. Suitable cosmetically acceptable excipients include, but are not limited to, humectants, emollients, oils, moisturizers, vitamins, fragrances, and/or plasticizers. Various exemplary cosmetically acceptable excipients as described in “Polish College: The Basics—An introduction to common nail polish ingredients.” by Doug Schoon, Dr. Vivian, B. Valenty, and Paul Brys Jun. 1, 2008, which is incorporated in relevant part herein.

Without limitation, exemplary cosmetically acceptable excipients include, but are not limited to, humectants, emollients, oils, moisturizers, vitamins, and/or fragrances. Other suitable excipients include plasticizers.

In some instances, the cosmetically acceptable excipients can be present in a concentration ranging from about 0.001 to 50 wt. % of the total weight of the photothermal nail composition, as well as individual concentration values or sub-ranges contained within.

### III. Methods of Removing Nail Coatings

Various methods are described herein that can be used in various applications in the nail care industry. The nail product(s) present in nail coatings are at least partially removable by exposure to a suitable removal stimulus, as described in the exemplary removal methods detailed below. The nail coatings also include a thermally responsive material, such as a shape changing, phase changing and/or thermally expandable material. In some instances the nail coating includes a plurality of layers, where at least the top layer is or includes a nail product, and at least one layer includes a thermally responsive material (such as a shape changing, phase changing and/or thermally expandable material. Optionally wherein the nail coating is on top of a bottom coating that directly contacts the subject's nail surface.

In one non-limiting instance, a method of removing a nail coating from a subject's nail includes the steps of:
(i) applying a photothermal nail composition, as described herein, on top of a nail coating;
wherein the photothermal nail composition is or forms a removal layer on top of the nail product; and
(ii) applying a removal stimulus to the removal layer for a sufficient amount of time to induce heat generation by the at least one photothermal additive and cause at least partial debonding, delamination, or detachment of the nail product from the subject's nail or from the interface with the thermally responsive material; or
   applying a removal stimulus to the removal layer for a sufficient amount of time to induce heat generation by the at least one photothermal additive and cause at least partial expansion, shape change, and/or phase change of the thermally responsive material.

In some instances, the nail coating, which may be or include a nail enhancement/ product, detaches from the partially expanded thermally responsive layer. In some other instances, a partially expanded thermally responsive material detaches from the nail surface. In yet other instances, the thermally expandable material rises/expands but does not cause spontaneous detachment at either interface but, nevertheless, facilitates removal by, such by peeling.

In some instances the nail coating is a multilayer nail coating that includes a nail product layer as the top layer, where the nail product layer is on top of one or more layers containing a thermally responsive material. Following application of the removal stimulus for a sufficient amount of time, a layer that is adjacent to the nail product layer and contains the thermally responsive material has undergone at least partial expansion, shape change, and/or phase change. Then the nail product layer can be removed at or below the interface between the nail product layer and a layer that contains the thermally responsive material, which has undergone at least partial expansion, shape change, and/or phase change.

In some instances, the removal stimulus is applied for a sufficient period of time to induce heat generation by the at least one photothermal additive that induces at least partial expansion, shape change, and/or phase change of the thermally responsive or shape-changing material and causes: (1) at least partial spontaneous debonding, delamination, lifting, separation, or detachment of the nail product from its interface with the at least partially expanded thermally responsive or shape-changing material, and/or (2) at least partial spontaneous debonding, delamination, lifting, separation, or detachment of the partially expanded thermally responsive or shape-changing material from its interface with the layer below, e.g., the nail surface or the optional bottom coating, and/or (3) further expansion of the at least partially expanded thermally responsive or shape-changing material that causes a weakening of at least the layer containing the material such that the layer that contains the at least partially expanded material (previously referred to as the layer containing the thermally responsive material) can be removed along with the nail product by mechanical force, peeling, and/or picking, and optionally wiping with or without solvent to remove any residue of the at least partially expanded material.

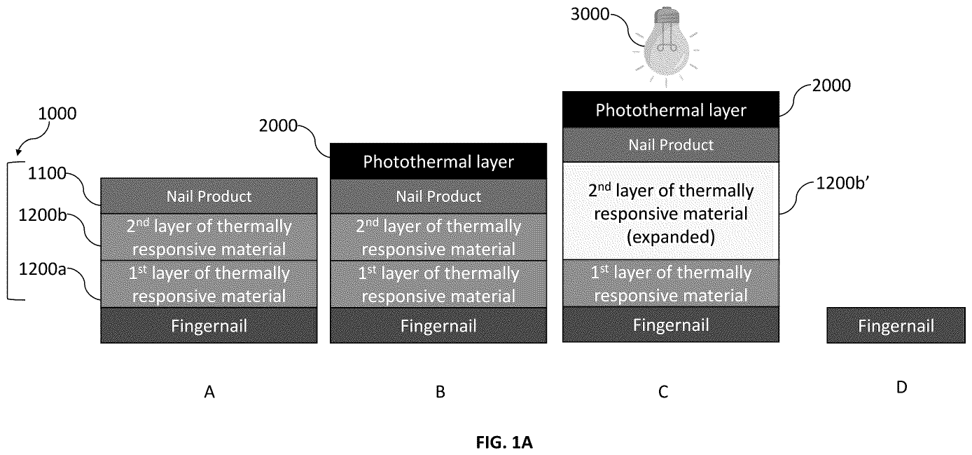

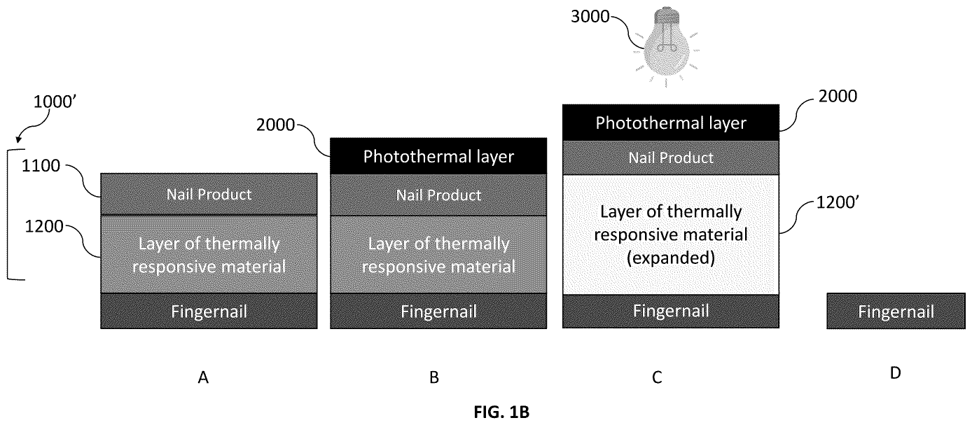

As shown in FIG. 1A, in one non-limiting example of a removal process, a fingernail has a multilayer coating 1000 having two or more layers containing a thermally responsive material(s) 1200a, 1200b, such as a shape changing, phase changing and/or thermally expandable material, and a nail product 1100 such as a gel, artificial nail, dip powder, or other nail enhancement thereon. When removal is desired, a photothermal layer 2000 is applied on top of top portion of the coating, i.e. the nail product 1100, and application of a removal stimulus 3000 causes heat generation by the photothermal layer which is conducted through the layers of the nail coating and causes expansion, shape change, and/or phase change of at least a portion of the thermally responsive material in the second layer 1200b′ causing rising, lifting, cracking, debonding, partial or full-delamination, and/or breakage of the nail product (i.e. the top portion of the nail coating) 1100 and facilitating the removal of the nail product 1100 along with the rest of the nail coating 1000 (see, e.g., FIG. 1A, part D).

A modified version of the removal process depicted in FIG. 1A is depicted in FIG. 1B. FIG. 1B depicts the same removal process as depicted in FIG. 1A, except that the nail coating 1000′ includes only a single layer 1200 containing a thermally responsive material and a nail product 1100, such as a multilayer nail gel or dip powder, artificial nail, or other nail enhancement thereon. As depicted in FIG. 1B, a single layer 1200 containing a thermally responsive material has a sufficient thickness (and a sufficient amount of the thermally responsive material) to facilitate the removal of the nail product 1100.

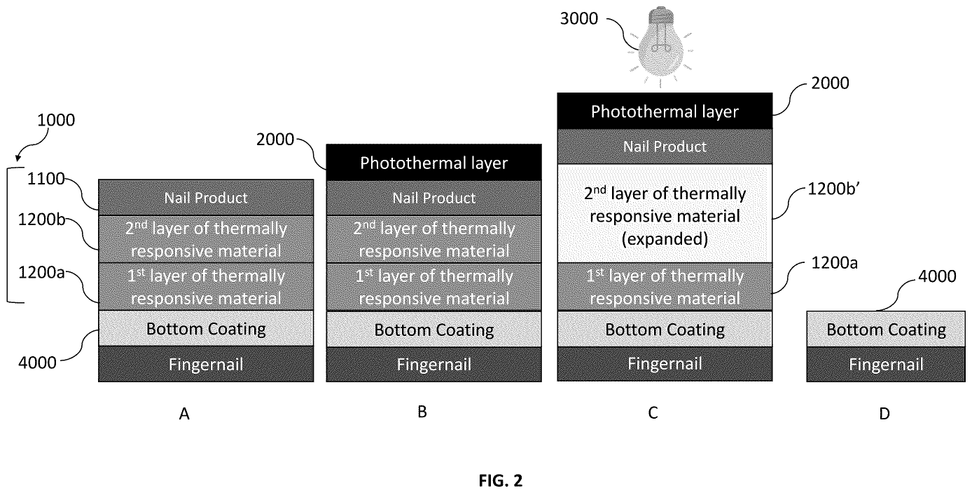

As shown in FIG. 2, in one non-limiting example of a removal process, a fingernail has a bottom coating 4000 and a multilayer nail coating 1000 thereon. When removal of the nail coating 1000 is desired, a photothermal layer 2000 is applied onto the top of the coating. Then a removal stimulus 3000 is applied, which causes heat generation by the photothermal layer 2000 and the generated heat is conducted through the layers of the nail coating and causes expansion, shape change, and/or phase change of at least a portion of the thermally responsive material, thereby causing rising, lifting, cracking, debonding, partial or full-delamination, and/or breakage of the nail product 1100 (i.e. the top portion of the nail coating) and facilitating the removal of the nail product 1100. The bottom coating 4000 typically remains attached or partially attached to the nail surface.

It is further understood that a modified version of the removal process depicted in FIG. 2 could involve a nail containing nail enhancement/product and only a single layer containing a thermally responsive material, instead of the two layers containing a thermally responsive material depicted in FIG. 2. In such a modification, the single layer containing a thermally responsive material has a sufficient thickness and a sufficient amount of the thermally responsive material to facilitate the removal of the nail enhancement/product.

In some instances of the above method, at least one layer of the plurality further includes a photothermal additive. For example, the nail product may include a photothermal additive in one or more of its layers. In some instances, some or all of the layers of the nail product do not contain any photothermal additives.

In another non-limiting instance, a method of removing a nail coating from a subject's nail includes the steps of:
(i′) applying a removal stimulus to the nail coating for a sufficient amount of time to induce heat generation by at least one photothermal additive in or on the nail coating and cause at least partial debonding, delamination, or detachment of a nail product of the nail coating from the subject's nail or from an interface with the thermally responsive material; or
   applying a removal stimulus to the nail coating for a sufficient amount of time to induce heat generation by at least one photothermal additive in or on the nail coating and cause at least partial expansion, shape change, and/or phase change of the thermally responsive material.

For the methods described herein, the subject can be a mammal, such as a human. In other instances, the subject can be avian or a reptile.

In some instances of the methods, the nail coating includes a plurality of layers. Optionally, at least two layers of the plurality include the thermally responsive material. In some other instances, only one layer of the plurality includes the thermally responsive material and it can be applied as a single layer.

As it relates to at least partial debonding, delamination, or detachment of the nail product, present in the top layer(s) of the nail coating, from the subject's nail, in some instances only the nail product of the top layer(s) debonds, delaminates, or detaches in step (ii) and (i′) of the methods. In such instances, other layers of the nail coating (including the thermally responsive material after expansion, shape change, and/or phase change) remain attached or substantially attached to the subject's nail after step (ii) or (i′) and can be removed, for example, by a further step of mechanical peeling, nicking, scratching, rubbing, rubbing with solvent, and/or mild washing with solvent, and may be followed by a further rinsing step after removal. In instances where a bottom coating is present, the bottom coating remains at least partially attached to the nail surface following step (ii) or (i′).

In some instances, applying a removal stimulus to the nail coating for a sufficient amount of time to induce heat generation by at least one photothermal additive in or on the nail coating and causes at least partial debonding, delamination, or detachment of a nail product of the nail coating from the subject's nail or from an interface with the thermally responsive material due to at least partial expansion, shape change, and/or phase change of the thermally responsive material.

In some instances, the methods described above result in the complete removal of the nail product in the top layer(s) of the nail coating following the application of the removal stimulus. In some other instances, the methods of removal described above result in a near total removal of the nail product following the application of the removal stimulus, where “near total” refers to removal of about 75% to 99.9% of the nail product from the nail (or from one or more layers of the nail coating thereon), as well as individual values and sub-ranges contained within the aforementioned range. In yet other instances, prior to applying the removal stimulus, a photothermal layer, such as a removal layer or top-most layer of the nail coating/enhancement is scratched, scored, or indented to facilitate removal.

The methods described herein facilitate the ability removal of a nail product, where the application of the removal stimulus allows for at least about 1% to 99.9% of the nail product in the top layer(s) of the nail coating and/or other layers of the nail coating to be removed, such as by peeling, nicking, scratching, rubbing, rubbing with solvent, and/or mild washing with solvent of the nail product, as well as individual values and sub-ranges contained within the aforementioned range.

### A. Exemplary Nail Enhancements and Nail Products

No particular limitation is placed on the nail product or nail enhancement that forms the top layer(s) of the nail coating. In some instances of the methods, the nail products are nail polishes, nail gels, a hybrid gel, a builder gel, an acrylic, a nail dip powder product, one or more artificial nails, nail art, or combinations thereof. In some instances, the nail product is a nail polish product, a nail gel product, or a nail dip powder product. Such nail products are known and commercially available. These are understood to be used to form a coating based on their instructions for use.

In some instances, the nail coating includes more than one layer that includes a nail product and at least the top layer is or includes a nail product. In other words, multiple nail product layers may be present in the nail coating in certain instances.

### B. Optional Bottom Coating

In some instances, the nail coating is on top of a bottom coating that directly contacts the subject's nail surface. Typically, the bottom coating remains at least partially attached on the nail surface following step (ii) or (i′) of the above methods.

1. Nail Composition for forming a Bottom Coating

Such a bottom coating can be formed from a suitable bottom coating nail composition. In some instances, the bottom coating nail composition includes a mixture of one or more active agent(s), such as monomers and optionally one or more crosslinkers; or the bottom coating nail composition includes a partially polymerized mixture of one or more monomers and optionally one or more crosslinkers; or the bottom coating nail composition includes one or more polymers. In some instances, the bottom coating is formed from the polymerization of an active agent, such as from the polymerization of vinyl monomers, α-lipoic acid, an ester of α-lipoic acid, an anhydride of α-lipoic acid, and/or an amide of α-lipoic acid.

In some other instances, the bottom coating nail composition includes a mixture of monomers where at least a portion of each of the monomers includes at least one cyclic ring including a disulfide or polysulfide bond therein, such as a 1,2-dithiolane, asparagusic acid, methyl asparagusic acid, α-lipoic acid, esters of α-lipoic acid (such as methyl lipoate and ethyl lipoate), amides of α-lipoic acid, multifunctional derivatives of α-lipoic acid (for example, a tris(dithiolane) formed by esterifying glycerin and α-lipoic acid), or a mixture thereof. In certain instances, the bottom coating nail composition includes α-lipoic acid. In some instances, the one or more monomers include one or more cyclic rings including at least one disulfide or polysulfide bond or oligomers/polymers derived therefrom are present in a concentration ranging from about 0.1 to 100 wt. %, 1 to 90 wt. %, 2 to 80 wt. %, 3 to 70 wt. %, 4 to 60 wt. %, 5 to 50 wt. %, 6 to 40 wt. %, 7 to 30 wt. %, 8 to 20 wt. %, 9 to 20 wt. %, or 10 to 20 wt. % of the total weight of the bottom coating nail composition before removal of any volatile solvent(s), as well as individual concentration values or sub-ranges contained within. In some other instances, the bottom coating nail composition includes one or more co-reactive vinyl monomers selected from acrylates, multifunctional acrylates (such as polyethylene glycol (200) diacrylate, polypropylene glycol (400) diacrylate, polytetramethylene glycol (650) diacrylate, tricyclodecane dimethanol diacrylate, ethoxylated bisphenol A diacrylate, ethoxylated trimethylolpropane triacrylate, ethoxylated glycerin triacrylate, pentaerythritol tri-or tetraacrylate, di(trimethylolpropane) tetraacrylate, dipentaerythritol polyacrylate, polypentaerythritol polyacrylate), diacrylates, triacrylates (such as trimethylolpropane triacrylate), tetraacrylates, pentaacrylates, hexaacrylates, methacrylates, dimethacrylates, trimethacrylates, tetramethacrylates, pentamethacrylates, hexamethacrylates, bis and tris(2-acryloxyethyl)isocyanurate, acrylamides, vinyl acetates, mono-alkenes, bis-alkenes, tris-alkenes, tetrakis-alkenes, pentakis-alkenes, polyurethane acrylates (such as supplied by Bomar), polyester acrylates (such as supplied by Bomar), polyether acrylates (such as supplied by Bomar), and mixtures thereof. In some instances, the one or more co-reactive vinyl monomers are present in a concentration ranging from about 0.1 to 10 wt. %, 0.1 to 5 wt. %, 0.1 to 2.5 wt. %, 0.1 to 1 wt. %, or 0.3 to 3 wt. % of the total weight of the bottom coating nail composition before removal of any volatile solvent(s), as well as individual concentration values or sub-ranges contained within.

In yet other instances, the bottom coating nail composition includes a reactive cyanoacrylate selected from methyl 2-cyanoacrylate, ethyl 2-cyanoacrylate, n-butyl cyanoacrylate, hexyl cyanoacrylate, 2-octyl cyanoacrylate, and mixtures thereof. In some instances, the sum of all reactive cyanoacrylate monomers is present in a concentration ranging from about 0.1 to 100 wt. % or 50 to 99 wt. % of the total weight of the bottom coating nail composition before polymerization and/or removal of any volatile solvent, as well as individual concentration values or sub-ranges contained within.

In some instances, the bottom coating nail composition includes one or more monomers that can undergo thiol-ene polymerization selected from a multifunctional thiol and a multifunctional alkene or multifunctional alkyne, or mixture thereof. In some instances, the reactive thiols and alkene monomers are present in a concentration ranging from about 0.1 to 100 wt. %, 1 to 100 wt. %, 5 to 100 wt. %, 10 to 100 wt. %, or 20 to 95 wt. % of the total weight of the bottom coating nail composition before polymerization and/or removal of any volatile solvent, as well as individual concentration values or sub-ranges contained within.

In some instances, the bottom coating nail composition includes reactive epoxides selected from one or more multifunctional epoxides and one or more multifunctional alcohols or amines, and mixtures thereof. In some instances, the reactive epoxide monomers are present in a concentration ranging from about 0.1 to 100 wt. %, 1 to 95 wt. %, 5 to 95 wt. %, 10 to 95 wt. %, 20 to 80 wt. %, or 30 to 70 wt % of the total weight of the bottom coating nail composition before polymerization and/or removal of any volatile solvent, as well as individual concentration values or sub-ranges contained within.

In some instances, the bottom coating is formed from the polymerization of an active agent, such as those containing vinyl monomers, and the bottom coating forms/acts as an adhesive, such as a glue.

In some instances, two or more distinct stacked bottom coatings may be formed on a nail surface, such that a first bottom coating is formed and then a second bottom coating is formed on top of the first. Without limitation, a first bottom coating may be formed by polymerization of α-lipoic acid, an ester of α-lipoic acid, an anhydride of α-lipoic acid, and/or an amide of α-lipoic acid, followed by a second bottom coating thereon formed by polymerization of vinyl monomers. Any combination of active agents, as described above, can be used to form such stacked bottom coatings.

### C. Thermally Responsive Materials

At least one layer of the plurality of layers in the nail coating includes a thermally responsive material, such as a shape changing, phase changing and/or thermally expandable material. A phase change can refer to melting of a material, e.g. from a solid or gel form to a liquid or flowable form. In some cases, at least two layers of the plurality include such a thermally responsive material. Optionally, the phase change involves changing from a solid or gel to forming a gas in a portion of a material or layer, resulting in the formation of a foam or partial foam material. Optionally, there may not be a phase change but a change in volume of the material upon exposure to heat, such as the expansion of gas contained within as the temperature increases. It is understood that the heat generated upon exposure of the photothermal additive to the removal stimulus is sufficient to activate (i.e., expand) the thermally responsive material in at least one portion of the at least one layer of the plurality of layers including the thermally responsive material. Such expansion, shape change, and/or phase change of the thermally responsive material typically causes lifting, peeling, debonding, and/or cracking of the nail product.

In some instances, the methods also include a step of scoring the nail enhancement and/or any top photothermal layer of the nail coating surface prior to step (ii) or (i′) to promote or define the cracking pattern upon expansion, shape change, and/or phase change, and facilitate removal of the nail product.

In some instances, at least a portion of the at least one layer of the plurality that includes the thermally responsive material can expand, when exposed to a sufficient temperature and for a sufficient period of time to form a closed-cell foam, an open-cell foam, or a combination thereof. The properties of such a foam play a role in thermal management and the comfort experienced by a user during the nail product removal processes described herein. A property of the foam is its thermal diffusivity α (units of m²/s), defined as α=k/(ρcₚ), where k is its thermal conductivity (units of W/(m·K)), ρ is the density of the material (units of kg/m³) and cₚ is the specific heat capacity at constant pressure (units of J/(kg·K)).

Thermal diffusivity is a material property that describes how rapidly heat spreads through a material. A thermally responsive material can be selected to absorb a sufficient amount of heat to activate (i.e., expand) but then become a foam with very low thermal diffusivity such that heat cannot spread further (or spreads very slowly) down through the foam into a user's nail to prevent user discomfort. Because thermal diffusivity depends on the ratio of thermal conductivity to specific heat capacity, one can choose a thermally responsive material that forms a foam with a low thermal conductivity and large specific heat capacity. For example, the thermal diffusivity of the thermally responsive material before foaming could be in the range of about 0.01×10⁻⁶m²/s to 0.2×10⁻⁶m²/s, as well as individual concentration values or sub-ranges contained within. The thermal diffusivity of the thermally responsive material after foaming could be in the range of about 0.001×10⁻⁶m²/s to 0.05×10⁻⁶m²/s, or 0.0005×10⁻⁶ m²/s to 0.03×10⁻⁶m²/s, as well as individual concentration values or sub-ranges contained within.

The formation of such foams results in lower thermal diffusivities of the expanded layer(s), as compared to prior to exposure to the thermal stimulus, that can act as a thermal management layer and function as an insulator, by dissipating, blocking, and/or absorbing a sufficient amount of heat to at least partly reduce the exposure of the subject's nail surface to high temperatures that would otherwise damage the natural nail surface on which the coating layer(s) are formed on. In some instances, use of thermal management additive(s) can prevent the nail surface from being exposed to temperatures greater than about 35° C. to 65° C., 38° C. to 62° C., or 40° C. to 60° C., 43° C. to 57° C., 45° C. to 55° C., and 47° C. to 53° C., as well as individual values or sub-ranges contained within the aforementioned ranges.

In some instances when exposed to the heat generated by the photothermal additive(s) when it is exposed to the removal stimulus, the at least one layer of the plurality that includes the thermally responsive material expands to form a foam. In some instances, the at least one layer of the plurality that includes the thermally responsive material that expands to form a foam, such as those described above, causes at least the nail product to at least partially or fully debond, delaminate, or detach from the rest of the nail coating while the expanded foam layer remains attached to the nail. In some instances, the at least partial or full debonding of the nail product is not completely spontaneous, but because the adhesion between the one or more nail product (top) layers and the thermally expanded foam becomes weak after foaming, the one or more nail product layers can be easily removed by mechanical force, such as peeling or scratching. Subsequently, the at least one layer that foamed and any other layer(s) beneath can be removed by mechanical force, such as peeling, picking, and/or scratching.

In some instances, the layer including the thermally responsive or shape changing material in the coating, on treatment with the removal stimulus, produces gradient foaming of the layer closest to the top of the coating exposed to the removal stimulus with the lower portion of the layer exhibiting no foaming or incomplete foaming without expansion of all of the thermally responsive or shape changing material contained within the lower portion of the layer. In other instances, where there are two or more layers including the thermally responsive material in the coating, the removal stimulus may only cause foaming of the layer(s) closest to the top of the coating exposed to the removal stimulus and lower portion(s) of the layer(s) may only exhibit no foaming or incomplete foaming without expansion of all of the thermally responsive material contained within these layer(s).

In some instances, the thermally responsive material includes a plurality of thermally expandable microspheres. Without limitation, such thermally expandable microspheres can be prepared by the encapsulation of a gaseous component in a suitable thermoplastic polymer shell. The gaseous or blowing component expands inside the polymer shell upon the application of a thermal energy stimulus for an effective period of time. The gaseous or blowing component can include, without limitation, one or more gas agents such as C₃-C₇ alkane gases, such as butane, isobutane, propane, heptane and the like, fluorinated alkanes, and mixtures thereof. Such gaseous components are liquid at room temperature after synthesizing the expandable microspheres and transition to the gas state at elevated temperatures.

In some instances, the plurality of thermally expandable particles are present in the at least one layer of the plurality that includes the thermally responsive material in a range from about 1 wt. % to 99 wt. %, 1 wt. % to 75 wt. %, 1 wt. % to 50 wt. %, 1 wt. % to 25 wt. %, 1 wt. % to 10 wt. %, 1 wt. % to 5 wt. %, 10 wt. % to 50 wt. %, 15 wt. % to 45 wt. %, 20 wt. % to 40 wt. %, 25 wt. % to 35 wt. %, or 30 wt. % to 40 wt. % of the total weight of the layer, as well as individual values or sub-ranges contained within the aforementioned ranges.

In some instances, exemplary suitable commercially available thermally expandable microspheres can be used which are sold under the name EXPANCEL® MICROSPHERES (Nouryon, Inc), and are formed of a thermoplastic vinyl resin polymer shell and core composed of alkane gas. EXPANCEL® MICROSPHERES offer a variety of microspheres with different expansion temperatures and properties. This includes, without limitation, EXPANCEL®551 DE, EXPANCEL® 920 DE, and EXPANCEL® 031 DU. Other commercially available thermally expandable microspheres include: DUALITE® (Henkel) such as DUALITE® M 6001 AE and DUALITE® M 7000 AE; MICROPEARL® (Matsumoto Yushi-Seiyaku Co., Ltd.) such as MICROPEARL® F 100D and MICROPEARL® F 80D; SPHERICEL® (Potters Industries), such as SPHERICEL@ 60P 40 and SPHERICEL® 110P8; and SUNSPHERE® (Sunjin Chemical) such as SUNSPHERE® H-33 and SUNSPHERE® H-51. Combinations of different types of thermally expandable microsphere particles may be used.

The thermally expandable microsphere particles can have any suitable average diameter. In some instances, the plurality of thermally expandable microspheres has an average particle diameter of between about 1 to 100 micrometers, 1 to 90 micrometers, 1 to 80 micrometers, 1 to 70 micrometers, 1 to 60 micrometers, 1 to 50 micrometers, 1 to 40 micrometers, 1 to 30 micrometers, 1 to 20 micrometers, or 1 to 10 micrometers, as well as individual values or sub-ranges contained within the aforementioned ranges. In some instances, the plurality of thermally expandable microspheres has an average particle diameter ranging from about 10 to about 16 micrometers or 10 to about 20 micrometers, as well as individual values or sub-ranges contained within the aforementioned range. In other instances, the plurality of thermally expandable microspheres has an average particle diameter ranging from about 25 to about 40 micrometers or about 20 to about 40 micrometers, as well as individual values or sub-ranges contained within the aforementioned range. The average particle diameter can be measured by various known methods, such as by dynamic light scattering using a commercial particle size analyzer, laser diffraction, and microscopy techniques.

In some instances, the thermally expandable microspheres may be conditioned, treated, and/or modified to decrease the temperature needed to activate a phase and/or shape transition or change the effective period of time required to induce a thermally induced phase and/or shape transition of the thermally expandable microspheres leading to their expansion on exposure to heating, as compared to the same particles in the absence of the conditioning, treatment, and/or modification.

In some instances, the thermally expandable microspheres undergo at least partial volume expansion at temperatures between about 30 to 120° C., or between 30 to 110° C., or between 30 to 100° C., or between 30 to 90° C., or between 35 to 85° C., or between 40 to 70° C., or between 45 to 60° C., or between 45° C. to 55° C., or between 50° C. to 60° C., or between 50° C. to 55° C., or between 45° C. to 50° C., or between 55° C. to 60° C., or between 60° C. to 70° C., or between 60° C. to 65° C., or between 65° C. to 70° C., or between 70° C. to 75° C., as well as individual values or sub-ranges contained within the aforementioned ranges. It is understood that there can be an interplay between time and heating temperature to cause sufficient expansion, meaning that in some instances longer times may be used for expansion at lower temperatures and shorter times may be used for expansion at higher temperatures. In some instances, expansion of the thermally expandable microspheres in the aforementioned ranges of temperatures occurs in time periods ranging from about 5 seconds to 15 minutes, 30 seconds to 15 minutes, 10 seconds to 10 minutes, 30 seconds to 10 minutes, 30 seconds to 5 minutes, 30 seconds to 3 minutes, 1 minute to 10 minutes, 1 minute to 5 minutes, or 1 minute to 2.5 minutes, as well as individual values or sub-ranges contained within the aforementioned ranges.

### D. Nail Coatings and Thermal Management Thereof

The nail coating can be formed of a single layer or multiple layers. The nail coating can include a plurality of layers where at least one layer includes a thermally responsive material and a top layer that is or includes a nail product or enhancement (i.e., a nail product layer) that may be formed on or is present on a nail surface. Alternatively, a single nail coating layer including a thermally responsive material that is or includes a nail product or enhancement may be formed on or is present on a nail surface. Such multilayered or single-layered nail coatings can be formed by various methods. Typically, the nail coatings to be removed by the methods described exclude the photothermal additive(s) described above which are applied as a separate photothermal coating at the time that removal of the nail coating is desired. However, there may be other instances where the nail coating includes a thermally responsive material and photothermal additive(s) therein in one or more layers of the coating and the photothermal additive(s) are also responsive to the removal stimulus.

In general, each layer in a multilayer nail coating is formed following a single application of a single composition. However, the nail product layer in a multilayer nail coating can be formed by a single application of a nail product composition that forms the nail product or multiple layers of the same or different compositions that form the resulting nail product. Thus, in a multilayered nail coating, the nail product is distinct from the other materials in the nail coating, but can be formed following multiple applications of the same or different compositions. For example, in some instances, the nail product layer can be on top of one or more layers containing a thermally responsive material.

In one non-limiting instance, a method of forming a multilayer nail coating on a subject's nail includes the steps of:
(i″) forming at least a first coating layer on a nail surface;
(ii″) optionally forming a second coating layer onto the at least first coating layer; wherein at least one of the first or the optional second coating layers includes a thermally responsive material (such as a shape changing, phase changing and/or thermally expandable material); and
(iii″) forming a top layer which is or includes a nail product on top of the first and optional second coating layers.

Typically, it is understood that forming multiple layers to provide a multilayered nail coating involves applying nail compositions and allowing for each respective composition to form a coating (such as by drying and/or curing) prior to applying another composition to form a subsequent layer on top.

In some instances, the thickness of each layer that contains a thermally responsive material is chosen to be about 1 micrometer to 500 micrometers, 10 micrometers to 400 micrometers, 30 micrometers to 300 micrometers, 50 micrometers to 250 micrometers, or 50 micrometers to 200 micrometers, as well as individual values or sub-ranges contained within the aforementioned ranges.

As noted above, the nail products can be a nail polish, nail gel, hybrid gel, builder gels, an acrylic, a nail dip powder product, one or more artificial nails, or any combination thereof. In some instances, the nail product is a nail polish product, a nail gel product, or a nail dip powder product. In other instances, the nail product is an artificial nail attached to the adjacent layer of the nail coating with glue that has similar chemistry as nail gel and/or nail dip powder products.

In some instances, the above method further includes a step of forming a bottom coating layer onto the nail surface prior to step (i″). Such bottom coatings are described above. In some cases, the bottom coating is optionally formed from the polymerization of an active agent selected from vinyl monomers, α-lipoic acid, an ester of α-lipoic acid, an anhydride of α-lipoic acid, or an amide of α-lipoic acid.

In some instances, a second coating layer is present. In some instances, both the first and the second coating layers include the same or a different thermally responsive material.

Exemplary thermally responsive materials are described above. In some instances, the first and optional second coating layers contain different thermally expandable materials. For example, the first coating layer may include EXPANCEL® 031 DU 40 and the second coating layer, when present, may include EXPANCEL® 043 DU 80 or the like. Such a combination may be used to improve the adhesion of a nail product, e.g., on the optional second coating layer, and/or improve expansion performance (e.g., extent of lifting, foaming, cracking) or decrease in thermal diffusivity when exposed to the removal stimulus. In some instances, one or more coating layers contain mixtures of different thermally expandable materials, such as a mixture of EXPANCEL® 031 DU 40 and EXPANCEL® 043 DU 80, or the like.

In some instances, the at least one layer of the plurality including the thermally responsive material expands to form a foam, when exposed to the removal stimulus. The thermally responsive material in the coating layer(s) can expand to form a closed-cell foam, an open-cell foam, or a combination thereof, as described above, when exposed to the removal stimulus.

Such coating layers including a thermally responsive material may be formed from a suitable nail composition which are applied onto a nail surface or onto a bottom coating.

In some non-limiting instances, such a nail composition includes a plurality of thermally expandable microspheres suspended (i.e., forms a suspension) in a cosmetically acceptable excipient such as a volatile solvent, a non-volatile diluent, or a combination thereof. In such instances, it is understood that the volatile solvent will evaporate to produce the coating layer. Without limitation, exemplary volatile solvents can be selected from acetone, ethyl acetate, propyl acetate, butyl acetate, ethanol, isopropanol, and combinations thereof. In some instances, the volatile solvent is ethanol or isopropanol. In other instances, the nail composition can be provided as a slurry of the plurality of wet thermally expandable microspheres (also referred to as a “wet cake”) that are combined with one or more solvents or carriers or a combination of at least one volatile solvent and at least one non-volatile diluent. In some instances, such a slurry includes the plurality of particles in acetone, ethyl acetate, propyl acetate, butyl acetate, ethanol, isopropanol, water, glycerin, propylene glycol, or combinations thereof, or the like. In some instances, the slurry includes about 1 to 99 wt. % of the wet thermally expandable microspheres (defined as the wet-cake mass of water plus the thermally expandable microspheres), 10 to 80 wt. % of thermally expandable microspheres, 20 to 70 wt. % of thermally expandable microspheres, 30 to 60 wt. % of thermally expandable microspheres, or 40 to 60 wt. % of thermally expandable microspheres, as well as individual values or sub-ranges contained within the aforementioned ranges.

In some instances, the plurality of thermally expandable microspheres are conditioned, treated, or modified before being applied to form a coating layer so that the temperature and period of time required to induce a thermally induced phase and/or shape transition at a given temperature is reduced, as compared to the same plurality of thermally expandable microspheres in the absence of such conditioning, treatment, or modification. For example, this pre-treatment might involve heating the particles at a specific temperature in an oven. In other instances, heating at a specified temperature may be performed in a closed chamber with a controlled atmosphere, such as in the presence of solvent vapor, for example, using glycerin, propylene glycol, alcohols, esters, or the like. Conditioning by thermal annealing may also be performed by submersing the plurality of thermally expandable microspheres in a solvent or mixture of solvents, optionally containing other solutes such as sodium chloride, potassium chloride, calcium carbonate, or the like, or a combination thereof. In some instances, the nail composition also includes one or more secondary additives therein, where the one or more secondary additives can lower the volume expansion temperature and/or volume expansion time and/or increase the stimulus-induced change in volume of the thermally expandable microspheres, as compared to the thermally expandable microspheres without the one or more secondary additives. In some instances, the secondary additive includes at least one plasticizing solvent having a boiling point of at least 100° C. at 1 atm of pressure, such as water, glycerin, triacetin, dimethylsulfoxide, sulfolane, Cyrene™, lactones (valerolactone, dodecalactone, undecalactone, decalactone, and the like), and eucalyptol. In some other instances, the one or more secondary additives are salts such as sodium chloride or potassium chloride, or combinations thereof.

In certain instances, the nail composition used to form any of the first, optional second coating layers, or the bottom coating each independently further include one or more thermal management additives that can dissipate, block, and/or absorb a sufficient amount of thermal energy to at least partly reduce the exposure of the subject's nail surface to high temperatures which could damage the natural nail surface on which the coating layer(s) are formed on. In some instances, use of the thermal management additive(s) can prevent the nail surface from being exposed to temperatures greater than about 35° C. to 65° C., 38° C. to 62° C., 40° C. to 60°C., 43° C. to 57° C., 45° C. to 55° C., and 47° C. to 53° C., as well as individual values or sub-ranges contained within the aforementioned ranges. Exemplary thermal management additives can be selected, without limitation, from pre-expanded microspheres (e.g., EXPANCEL® DE products), ceramic particles, hollow ceramic particles, yttria-stabilized zirconia particles, hollow yttria-stabilized zirconia particles, mullite, alumina, cerium oxide, rare earth zirconates, rare earth oxides, metal-glass composites, or combinations thereof. In some instances, the total concentration of the one or more thermal management additives are present at ranges from about 0.0001 wt. % to 60 wt. % of the total weight of the nail composition, such as from about 0.001 wt. % to 50 wt. %, from 0.01 wt. % to 50 wt. %, from 0. 1 wt % to 40 wt. %, from 1 wt. % to 30 wt %, from 1 wt. % to 20 wt. %, from 1 wt % to 10 wt. %, from 1 wt. % to 5 wt. %, as well as individual values or sub-ranges contained within.

### E. Removal Stimulus

The removal stimulus can be applied from any suitable source. In some instances, the removal stimulus is produced by an electronic device including one or more light sources, such as light-emitting diodes (LEDs), infrared emitters, intense pulsed light (IPL), an arc lamp, or a laser which produce the removal stimulus. In other instances, the light source is an intense pulsed light source, such as that produced by a xenon arc lamp.

In some instances, the removal stimulus includes one or more wavelengths in the visible to near-infrared spectrum range (about 400 nm to about 2500 nm, as well as individual values or sub-ranges contained within the aforementioned ranges) and can encompass emission of broad-band white light. In some instances, the removal stimulus includes broadband light sources that comprise of all wavelengths in the visible to near-infrared spectrum range. In some instances, the removal stimulus includes one or more wavelengths in the near-infrared spectrum range (about 700 nm to about 2500 nm, as well as individual values or sub-ranges contained within the aforementioned ranges), such as at about 850 nm. In some instances, the one or more light sources used produce one or more wavelengths with a full-width at half maximum of about 200 nm or less or about 100 nm or less; and/or contain a mixture of at least two or more of the LEDs or infrared emitters having different maximum wavelengths and different full-width at half maximum values.

In some instances, the removal stimulus is applied for a period of time ranging from about 5 seconds to 15 minutes, 30 seconds to 15 minutes, 10 seconds to 10 minutes, 30 seconds to 10 minutes, 30 seconds to 5 minutes, 1 minute to 10 minutes, 1 minute to 10 minutes, 1 minute to 5 minutes, or 1 minute to 2.5 minutes.

As described above, the removal stimulus causes heating of the photothermal nail composition. During exposure to the removal stimulus, the temperature as measured at the top surface of the removal layer, for example by a thermal camera such as a P2W Thermal Camera, can be significantly higher than that temperature felt by the user through their nail at the bottom of the nail coating. For example, after 30 seconds of exposure to the removal stimulus, the temperature measured at the top surface could be in the range of about 60° C. to 250° C., 70° C. to 200° C., 80° C. to 175° C., 90° C. to 150° C., 100° C. to 150° C., and the like.

The precise temperature measured in this way will depend on the photothermal nail composition as well as the exposure conditions, such as the exposure time, intensity of the removal stimulus, and atmosphere of the room, among other possible contributing factors.

A benefit of applying the removal stimulus to a photothermal nail composition is that absorption and corresponding heat generation occur primarily at the topmost layer of the nail coating; there is no need to penetrate the removal stimulus deep into the nail coating, which would necessitate the design of special nail products that allow for the transmission of the removal stimulus. Nor is there a need to incorporate the photothermal agent into the optional bottom coat, the thermally expandable layer(s), or any other part of the nail coating except for the very top layer. Because photothermal agents are often dark in color, having to incorporate them into the nail product itself or layers below in the nail coating could interfere with the desired look of a nail product during wear and use.

### IV. Kits

Kits useful for practicing the methods described above, and photothermal nail compositions used therein, are also disclosed. For example, kits are provided for nail care applications, such as removing nail coatings and applying photothermal nail compositions for removal of such nail coatings on-demand by way of application of a suitable removal stimulus.

In one instance, a kit includes the photothermal nail composition as described herein. In another instance, a kit includes the photothermal nail composition as described herein; and a nail product composition including a thermally responsive material. Such nail compositions including a thermally responsive material are described above.

In yet another instance, a kit includes:
a nail composition to form a bottom coating;
a nail composition including a thermally responsive material; and
a photothermal nail composition.

Such nail compositions including a thermally responsive material and nail compositions for forming a bottom coating are described above. Suitable photothermal nail compositions are also described above.

In some instances, the kits described can further include one or more nail product compositions for forming a nail product, such as to form a nail product layer(s). Such nail product compositions can be selected from nail polishes, nail gels, a hybrid gel, a builder gel, an acrylic, artificial nails, a nail dip powder product, one or more artificial nails, or combinations thereof. In some instances, the nail product is a nail polish product and the kits described above further include a nail polish composition. In some instances the nail product is a nail gel product, and the kits described above further include a gel base composition, a color composition, and/or a top coat composition for forming the nail gel product. In some instances the nail product is a nail dip powder product, and the kits described above further include a base coat composition, a colored powder, a clear powder, a sealing composition, and/or a top coat composition.

In some instances, the kits described further include instructions and/or a device that is capable of producing a removal stimulus.

In some instances, the kits described further include a buffing block, which can be used to lightly roughen the nail before application of a nail coating; a dehydrating agent, such as isopropanol, which can be used to prepare the nail for a nail coating; a tool configured to push back the cuticle, which can be used to prepare the nail for applying a nail coating thereon; and/or a tool to score the nail product, which can be used before or after applying the photothermal nail composition to direct cracking of the nail product during the removal process.

The disclosed methods for removing nail coatings, photothermal nail compositions, and kits can be further understood by reference to the following numbered paragraphs:

Paragraph 1. A method of removing a nail coating, comprising a thermally responsive material and a nail product, from a subject's nail, the method comprising the steps of:
(i′) applying a removal stimulus to the nail coating for a sufficient amount of time to induce heat generation by at least one photothermal additive in or on the nail coating and cause at least partial debonding, delamination, or detachment of the nail product from the subject's nail or from an interface with the thermally responsive material; or
applying a removal stimulus to the nail coating for a sufficient amount of time to induce heat generation by at least one photothermal additive in or on the nail coating and cause at least partial expansion, shape change, and/or phase change of the thermally responsive material.

Paragraph 2. The method of paragraph 1, wherein the nail coating comprises a plurality of layers;
wherein at least the top layer of the plurality is or comprises the nail product;
wherein at least one layer of the plurality comprises the thermally responsive material;
optionally wherein the nail coating is on top of a bottom coating that directly contacts the subject's nail surface; and optionally wherein the at least one photothermal additive is on top of the top layer of the plurality.

Paragraph 3. A method of removing a nail coating, comprising a thermally responsive material and a nail product, from a subject's nail, the method comprising the steps of:
(i) applying a photothermal nail composition, comprising at least one photothermal additive, on top of the nail coating;
wherein the photothermal nail composition is or forms a removal layer on top of the nail product; and
(ii) applying a removal stimulus to the removal layer for a sufficient amount of time to induce heat generation by the at least one photothermal additive and cause at least partial debonding, delamination, or detachment of the nail coating from the subject's nail or from an interface with the thermally responsive material; or
applying a removal stimulus to the removal layer for a sufficient amount of time to induce heat generation by the at least one photothermal additive and cause at least partial expansion, shape change, and/or phase change of the thermally responsive material.

Paragraph 4. The method of paragraph 3, wherein the nail coating comprises a plurality of layers; wherein at least the top layer of the plurality is or comprises the nail product;
wherein at least one layer of the plurality comprises the thermally responsive material;
   optionally wherein the nail coating is on top of a bottom coating that directly contacts the subject's nail surface; and
   optionally wherein the photothermal nail composition further comprises:
a cosmetically acceptable carrier and/or matrix;
wherein the photothermal additive is selected from the group consisting of a carbon material, an organic dye, an inorganic compound, an inorganic element, an inorganic alloy, and combinations thereof; and
wherein the at least one photothermal additive is present at a concentration ranging from about 0.001 wt. % to 99 wt. % of the total weight of the photothermal nail composition.

Paragraph 5. The method of any one of paragraphs 1-4, wherein the nail product is a nail polish, a nail gel product, a nail dip powder product, an artificial nail product, nail art, or a combination thereof.

Paragraph 6. The method of any one of paragraphs 2-5, wherein at least two layers of the plurality comprise the thermally responsive material.

Paragraph 7. The method of any one of paragraphs 2-6, wherein the bottom coating is present and the bottom coating is formed from the polymerization of an active agent selected from vinyl monomers, α-lipoic acid, an ester of α-lipoic acid, an anhydride of α-lipoic acid, or an amide of α-lipoic acid.

Paragraph 8. The method of paragraph 7, wherein the bottom coating remains at least partially attached on the nail surface following step (ii) or (i′).

Paragraph 9. The method of any one of paragraphs 1-8, wherein the thermally responsive material comprises a plurality of thermally expandable microspheres.

Paragraph 10. The method of paragraph 9, wherein the thermally expandable microspheres undergo at least partial volume expansion at temperatures between about 30° C. to 80° C., or between 35° C. to 75° C., or between 40° C. to 65° C., or between 45° C. to 60° C., or between 45° C. to 55° C., or between 50° C. to 60° C., or between 50° C. to 55° C., or between 45° C. to 50° C., or between 55° C. to 60° C., or between 60° C. to 70° C., or between 60° C. to 65° C., or between 65° C. to 70° C., or between 70° C. to 75° C.

Paragraph 11. The method of any one of paragraphs 9-10, wherein the plurality of thermally expandable particles is present in the at least one layer of the plurality comprising the thermally responsive material and are present in a range from about 1 to 75 wt. % of the total weight of the at least one layer.

Paragraph 12. The method of any one of paragraphs 1-11, wherein the at least one photothermal additive present generates heat upon exposure to a removal stimulus producing an increase in temperature in the composition of by at least about 5° C., 10° C., 15° C., 20° C., 25° C., 30° C., 35° C., 40° C., 45° C., 50° C., 60° C., 70° C., 80° C., 90° C., 100° C., 110° C., 120 ° C., 130° C., 140° C., 150° C., 160° C., 170° C., or 180° C. following at least about 30 seconds to about 5 minutes of exposure to the removal stimulus, as compared to the composition prior to exposure to the removal stimulus.

Paragraph 13. The method of any one of paragraphs 1-12, wherein the heat generated is sufficient to expand or at least partially expand the thermally responsive material in the nail coating,
optionally wherein at least a portion of the at least one layer of the plurality comprising the thermally responsive material expands to form a closed-cell foam, an open-cell foam, or combination thereof, with a lower thermal diffusivity than the at least one layer prior to exposure to the removal stimulus.

Paragraph 14. The method of paragraph 13, wherein the at least partial expansion of the thermally responsive material causes lifting, cracking, debonding, partial or full-delamination, and/or breaking of the nail product.

Paragraph 15. The method of any one of paragraphs 1-14, wherein the method comprises a step of scoring the nail coating surface prior to step (ii) or (i′).

Paragraph 16. The method of any one of paragraphs 1-15, wherein the removal stimulus comprises one or more wavelengths in the visible to near-infrared spectrum range (about 400 nm to about 2500 nm).

Paragraph 17. The method of paragraph 16, wherein the removal stimulus comprises one or more wavelengths in the near-infrared spectrum range (about 700 nm to about 2500 nm), such as at about 850 nm.

Paragraph 18. The method of any one of paragraphs 1-17, wherein the removal stimulus is produced by a device comprising one or more light sources, such as light-emitting diodes (LEDs), infrared emitters, intense pulsed light (IPL), an arc lamp, or a laser.

Paragraph 19. The method of paragraph 18, wherein the one or more light sources produce wavelengths with a full-width at half maximum of about 100 nm or less; and/or contains a mixture of at least two or more of the LEDs or infrared emitters having different maximum wavelengths and different full width at half maximum values.

Paragraph 20. The method of any one of paragraphs 1-19, wherein the removal stimulus is applied for a period of time ranging from about 5 seconds to 15 minutes, 30 seconds to 15 minutes, 10 seconds to 10 minutes, 30 seconds to 10 minutes, 30 seconds to 5 minutes, 1 minute to 10 minutes, 1 minute to 10 minutes, 1 minute to 5 minutes, or 1 minute to 2.5 minutes.

Paragraph 21. A photothermal nail composition comprising:
at least one photothermal additive; and
a cosmetically acceptable carrier and/or matrix;
wherein the photothermal additive is selected from the group consisting of a carbon material, an organic dye, an inorganic compound, an inorganic element, an inorganic alloy, and combinations thereof;
   wherein the at least one photothermal additive is present at a concentration ranging from about 0.001 wt. % to 99 wt. % of the total weight of the photothermal nail composition; and
wherein the at least one photothermal additive present generates heat upon exposure to a removal stimulus producing an increase in temperature in the composition of by at least about 5° C., 10° C., 15° C., 20° C., 25° C., 30° C., 35° C., 40° C., 45° C., 50° C., 60° C., 70° C., 80° C., 90° C., 100° C., 110° C., 120° C., 130° C., 140° C., 150° C., 160° C., 170° C., or 180° C. following at least about 30 seconds to about 5 minutes of exposure to the removal stimulus, as compared to the composition prior to exposure to the removal stimulus.

Paragraph 22. The photothermal nail composition of paragraph 21, wherein the cosmetically acceptable carrier and/or matrix is selected from the group consisting of a solvent, a lacquer, monomers, film-forming polymers, and combinations thereof.

Paragraph 23. The photothermal nail composition of paragraph 22, wherein:
the solvent is selected from the group consisting of water; acetone; esters such as ethyl acetate, propyl acetate, and butyl acetate; linear and branched alcohols, such as ethanol, propanol, isopropanol, hexanol, and the like; aromatic or cyclic alcohols, such as benzyl alcohol, cyclohexanol, and the like; saturated C12 to C30 fatty alcohols, such as lauryl alcohol, myristyl alcohol, cetyl alcohol, stearyl alcohol, behenyl alcohol, and combinations thereof;
the monomers are selected from the group consisting of acrylates, methacrylates, acrylamides, cyanoacrylates, styrenes, epoxies, dithianes, and dithiolanes; and/or
the film-forming polymers are selected from the group consisting of nitrocellulose, cellulose acetate, cellulose acetate butyrate, ethyl cellulose, hydroxypropyl cellulose, other substituted cellulose derivatives, acrylates copolymer, methacrylates copolymer, dimethicone, substituted dimethicones, polyvinyl butyral, polyurethane resins, and tosylamide/formaldehyde resin.

Paragraph 24. The photothermal nail composition of paragraph 21, wherein the matrix is or comprises an adhesive and the photothermal nail composition is in the form of a sticker.

Paragraph 25. The photothermal nail composition of paragraph 21, wherein the cosmetically acceptable carrier or matrix can be evaporated or cured to form a coating.

Paragraph 26. The photothermal nail composition of any one of paragraphs 21-25, wherein the organic dye is selected from the group consisting of Epolin Epolight® (such as Epolight® 1125, Epolight® 3801, Epolight® 5588, Epolight® 5396, Epolight® 5393, donor-acceptor Stenhouse adducts, azobenzene derivatives, perylene diimide, polymer dyes, and combinations thereof.

Paragraph 27. The photothermal nail composition of any one of paragraphs 21-26, wherein the carbon material is selected from the group consisting of carbon black, graphene, graphene oxide, reduced graphene oxide, single-walled carbon nanotubes, multi-walled carbon nanotubes, acetylene black, graphite, activated carbon, carbon nanofibers, and combinations thereof.

Paragraph 28. The photothermal nail composition of any one of paragraphs 21-27, wherein the inorganic compound, inorganic element, or inorganic alloy is selected from the group consisting of indium-tin oxide (ITO), mica, titanium nitride, iron oxide, plasmonic particles (such as gold nanoparticles, gold nanorods, silver sulfide quantum dots, and silver nanoparticles), and combinations thereof.

Paragraph 29. The photothermal nail composition of any one of paragraphs 21-28, wherein the concentration of the photothermal additive ranges from about 0.1 wt. % to 99 wt. % of the total weight of the photothermal nail composition.

Paragraph 30. The photothermal nail composition of any one of paragraphs 21-28, wherein the concentration of the photothermal additive ranges from about 0.2 wt. % to 30 wt. % of the total weight of the photothermal nail composition.

Paragraph 31. The photothermal nail composition of any one of paragraphs 21-28, wherein the concentration of the photothermal additive ranges from about 0.5 wt. % to 20 wt. % of the total weight of the photothermal nail composition.

Paragraph 32. The photothermal nail composition of any one of paragraphs 21-28, wherein the concentration of the photothermal additive ranges from about 1 wt. % to 10 wt. % of the total weight of the photothermal nail composition.

Paragraph 33. The photothermal nail composition of any one of paragraphs 21-28, wherein the concentration of the photothermal additive ranges from about 2 wt. % to 5 wt. % of the total weight of the photothermal nail composition.

Paragraph 34. The photothermal nail composition of any one of paragraphs 21-28, wherein the photothermal nail composition further comprises at least one cosmetically acceptable excipient optionally selected from humectants, emollients, oils, moisturizers, vitamins, fragrances, and/or plasticizers.

Paragraph 35. A method of forming a multilayer nail coating on a subject's nail, the method comprising the steps of:
(i″) forming at least a first coating layer on a nail surface;
(ii″) optionally forming a second coating layer onto the at least first coating layer; wherein at least one of the first or the optional second coating layers comprises a thermally responsive material; and
(iii″) forming a top layer which is or comprises a nail product on top of the first and optional second coating layers.

Paragraph 36. The method of paragraph 35, wherein the nail product is a nail polish product, a nail gel product, an acrylic product, a nail dip powder product, an artificial nail product, nail art, or a combination thereof.

Paragraph 37. The method of any one of paragraphs 35-36, wherein the method further comprises a step of forming a bottom coating onto the nail surface prior to step (i″); and wherein the bottom coating is optionally formed from the polymerization of an active agent selected from vinyl monomers, α-lipoic acid, an ester of α-lipoic acid, an anhydride of α-lipoic acid, or an amide of α-lipoic acid.

Paragraph 38. The method of any one of paragraphs 35-37, wherein the second coating layer is present.

Paragraph 39. The method of any one of paragraphs 35-38, wherein the first and the second coating layers comprise the thermally responsive material.

Paragraph 40. A kit comprising the photothermal nail composition of any of paragraphs 21-34.

Paragraph 41. A kit comprising the photothermal nail composition of any of paragraphs 21-34; and
one or more compositions for forming a nail coating comprising a thermally responsive material.

Paragraph 42. A kit comprising:
a nail composition to form a bottom coating;
a nail composition comprising a thermally responsive material; and
the photothermal nail composition of any of paragraphs 21-34.

Paragraph 43. The kit of any one of paragraphs 40-42, further comprising one or more compositions for forming a nail product.

Paragraph 44. The kit of paragraph 43, wherein the nail product is a nail polish product, a nail gel product, a nail dip powder product, an artificial nail product, a nail art product, or a combination thereof.

Paragraph 45. The kit of any one of paragraphs 40-44, wherein the kit further comprises instructions and/or a device that is capable of producing a removal stimulus.

Paragraph 46. The kit of any one of paragraphs 40-45, wherein the kit further comprises a buffing block, a dehydrating agent such as isopropanol, a tool configured for manual use to push back the cuticle of a nail, and/or a tool to configured for scoring the nail product.

The disclosed methods for removing nail coatings, photothermal nail compositions, and kits can be further understood by reference to the following non-limiting Examples.

### EXAMPLES

### Example 1: Exemplary Nail Product Removal Processes

### Optional Bottom Coat:

A curable bottom coat was formulated with α-lipoic acid (15 wt %), trimethylolpropane triacrylate (1 wt %), and phenylbis(2,4,6-trimethylbenzoyl)phosphine oxide (3 wt %) in ethyl acetate or isopropanol.

### Thermally Responsive Material:

A paintable formulation containing a thermally responsive material contained a-Lipoic acid, lauryl acrylate; and thermally expandable microspheres (50% wt/wt), in a paintable formulation after stirring.

### Photothermal Nail Composition:

A commercially available nail lacquer (OPI Nail Lacquer Top Coat, Clear Nail Polish Top Coat) was mixed with 2 wt % short multiwalled carbon nanotubes (XFNANO XFM 04, OD 5 -15 nm, L=0.5-2 micrometers), resulting in a dark black, relatively well-dispersed viscous mixture.

### Application of the Nail Coating Shown in FIG. 1A (Part A)

### A Gel Nail Product:

The fingernail was prepped by light buffing and cleansing with isopropanol. After evaporation of the isopropanol, a first layer 1200a of thermally expandable material was applied with a small nail brush and cured for 60 seconds under 415 nm or 365 nm light. A second layer 1200b of thermally expandable material was applied in the same way. After curing, a commercially available nail gel product was applied by sequentially painting and curing a layer of (1) DND DC 800 gel base, (2) DND DC 140 Khaki Rose, (3) DND DC 140 Khaki Rose, (4) DND DC 900 top coat. Each layer was cured for 60 seconds under 415 nm light prior to applying the next layer. After application, the nail product 1100 was observed to be robust and performed as it usually would in the absence of the thermally expandable material.

### A Dip Nail Product:

The fingernail was prepped by light buffing and cleansing with isopropanol. After evaporation of the isopropanol, a first layer of thermally expandable material 1200a was applied with a small nail brush and cured for 60 seconds under 415 nm light. A second layer 1200b of thermally expandable material was applied in the same way. After curing, a commercially available nail dip product (Kiara Sky Professional Nails Dip Essential) was applied by (1) applying the #2 base coat and quickly dipping the finger into natural powder, (2) applying the #2 base coat and quickly dipping the finger into a colored powder, (3) applying the #2 base coat and quickly dipping the finger into a colored powder again, (4) applying the #2 base coat and quickly dipping the finger into a clear powder, (5) applying the #3 seal protect and letting the coating dry/harden for about 1 minute, and (6) applying the #4 top coat and letting it dry for 7 minutes. After application, the nail product 1100 was mechanically hard as usual (i.e. in the absence of the thermally expandable material).

### Application of the Nail Coating Shown in FIG. 2 (Part A)

The fingernail was prepped by light buffing and isopropanol. After evaporation of the isopropanol, the bottom coating was applied by brushing. After evaporation of the ethyl acetate, the bottom coating 4000 was cured for 60 seconds under 415 nm light. Either a gel or dip product 1100 was applied in the same way as described above (with respect to FIG. 1, part A) on the cured bottom coat.

### Application of the Photothermal layer shown in FIG. 1A (Part B) and FIG. 2 (Part B)

The nail coatings described above were coated with the photothermal nail composition by painting with a brush. After about 3-5 min, the resulting photothermal layer 2000 was black in color and solid to the touch and was located on top of the gel or dip coating, i.e. the nail product 1100.

### Expansion of the Thermally Responsive Layer Shown in FIG. 1A (Part C) and FIG. 2 (Part C)

The photothermal layer was exposed a removal stimulus 2000 in the form of LED lights with a maximum wavelength of about 850 nm for 1 to 5 minutes, which resulted in cracking of the nail product 1100 and/or visible lifting/whitening of part of the thermally expandable material layer 1200b′. After stopping exposure to the light and cooling to room temperature, the nail product 1100, i.e. the gel or dip coating, was easy to detach from the expanded layer 1200b′ by light peeling. The expanded material (and underlying unexpanded material 1200a and optional bottom coat 4000) remained attached to the finger even after the nail product was removed. The expandable material layer may be visibly stratified by eye into two distinct layers: a top-portion 1200b′ (nearest the now-removed nail product) that was white and foamy, and a layer underneath that remained unexpanded with a more pronounced yellow tint 1200a.

### Final Cleanup in FIG. 1A (Part D) and FIG. 2 (Part D)

The still-attached first layer of expandable material 1200a and still-attached second layer of expanded material 1200b′ were removed by a combination of mechanical peeling, mechanical rubbing, and rubbing/dabbing with a paper towel soaked with acetone or ethyl acetate. Following the process depicted in FIG. 1A, the resulting nail was a healthy-looking nail that remained largely unaffected by the removal process. Following the process depicted in FIG. 2, i.e. where a bottom coating 4000 was applied directly to the nail prior to applying the multilayer nail coating, the nail was substantially intact after the removal process was complete, even after rubbing/dabbing with a paper towel soaked with acetone or ethyl acetate.

While the process described above utilizes two layers of thermally expandable material 1200a 1200b, the processes depicted in FIGS. 1A and 2 can be modified to utilize a single layer of thermally expandable material. For example, FIG. 1B depicts a modified version of FIG. 1A utilizing a single layer of thermally expandable material. The process depicted in FIG. 2 and described above can be modified in a similar manner to utilize a single layer of thermally expandable material (i.e. only a single layer thermally expandable material is applied).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific instances of the invention described herein. Such equivalents are intended to be encompassed by the following claims.

1. A method of removing a nail coating, comprising a thermally responsive material and a nail product, from a subject's nail, the method comprising the steps of:
(i′) applying a removal stimulus to the nail coating for a sufficient amount of time to induce heat generation by at least one photothermal additive in or on the nail coating and cause at least partial debonding, delamination, or detachment of the nail product from the subject's nail or from an interface with the thermally responsive material; or
applying a removal stimulus to the nail coating for a sufficient amount of time to induce heat generation by at least one photothermal additive in or on the nail coating and cause at least partial expansion, shape change, and/or phase change of the thermally responsive material;
optionally wherein the nail product is a nail polish, a nail gel product. a nail dip powder product. an artificial nail product, nail art, or a combination thereof.

2. The method of claim 1, wherein the nail coating comprises a plurality of layers;
wherein at least the top layer of the plurality is or comprises the nail product;
wherein at least one layer of the plurality comprises the thermally responsive material;
optionally wherein the nail coating is on top of a bottom coating that directly contacts the subject's nail surface; and
optionally wherein the at least one photothermal additive is on top of the top layer of the plurality.

3. A method of removing a nail coating, comprising a thermally responsive material and a nail product, from a subject's nail, the method comprising the steps of:
(i) applying a photothermal nail composition, comprising at least one photothermal additive, on top of the nail coating;
wherein the photothermal nail composition is or forms a removal layer on top of the nail product; and
(ii) applying a removal stimulus to the removal layer for a sufficient amount of time to induce heat generation by the at least one photothermal additive and cause at least partial debonding, delamination, or detachment of the nail coating from the subject's nail or from an interface with the thermally responsive material; or
applying a removal stimulus to the removal layer for a sufficient amount of time to induce heat generation by the at least one photothermal additive and cause at least partial expansion, shape change, and/or phase change of the thermally responsive material:
optionally wherein the nail product is a nail polish, a nail gel product, a nail dip powder product, an artificial nail product, nail art, or a combination thereof.

4. The method of claim 3, wherein the nail coating comprises a plurality of layers;
wherein at least the top layer of the plurality is or comprises the nail product;
wherein at least one layer of the plurality comprises the thermally responsive material;
optionally wherein the nail coating is on top of a bottom coating that directly contacts the subject's nail surface; and
optionally wherein the photothermal nail composition further comprises:
a cosmetically acceptable carrier and/or matrix;
wherein the photothermal additive is selected from the group consisting of a carbon material, an organic dye, an inorganic compound, an inorganic element, an inorganic alloy, and combinations thereof; and
wherein the at least one photothermal additive is present at a concentration ranging from about 0.001 wt. % to 99 wt. % of the total weight of the photothermal nail composition.

5. (canceled)

6. (canceled)

7. The method of claim 2, wherein the bottom coating is present and the bottom coating is formed from the polymerization of an active agent selected from vinyl monomers, α-lipoic acid, an ester of α-lipoic acid, an anhydride of α-lipoic acid, or an amide of α-lipoic acid.

8. The method of claim 4, wherein the bottom coating is present and the bottom coating is formed from the polymerization of an active agent selected from vinyl monomers, α-lipoic acid, an ester of α-lipoic acid, an anhydride of α-lipoic acid, or an amide of α-lipoic acid.

9. The method of claim 1, wherein the thermally responsive material comprises a plurality of thermally expandable microspheres that optionally undergo at least partial volume expansion at temperatures between about 30° C. to 80° C., or between 35° C. to 75° C., or between 40° C. to 65° C., or between 45° C. to 60° C., or between 45° C. to 55° C., or between 50° C. to 60° C., or between 50° C. to 55° C., or between 45° C. to 50° C., or between 55° C. to 60° C., or between 60° C. to 70° C., or between 60° C. to 65° C., or between 65° C. to 70° C., or between 70° C. to 75° C.

10. The method of claim 3, wherein the thermally responsive material comprises a plurality of thermally expandable microspheres that optionally undergo at least partial volume expansion at temperatures between about 30° C. to 80° C., or between 35° C. to 75° C., or between 40° C. to 65° C., or between 45° C. to 60° C., or between 45° C. to 55° C., or between 50° C. to 60° C., or between 50° C. to 55° C., or between 45° C. to 50° C., or between 55° C. to 60° C., or between 60° C. to 70° C., or between 60° C. to 65° C., or between 65° C. to 70° C., or between 70° C. to 75° C.

11. (canceled)

12. (canceled)

13. The method of claim 1 wherein the heat generated is sufficient to expand or at least partially expand the thermally responsive material in the nail coating, optionally wherein at least a portion of the at least one layer of the plurality comprising the thermally responsive material expands to form a closed-cell foam, an open-cell foam, or combination thereof, with a lower thermal diffusivity than the at least one layer prior to exposure to the removal stimulus.

14. The method of claim 3, wherein the heat generated is sufficient to expand or at least partially expand the thermally responsive material in the nail coating, optionally wherein at least a portion of the at least one layer of the plurality comprising the thermally responsive material expands to form a closed-cell foam, an open-cell foam, or combination thereof, with a lower thermal diffusivity than the at least one layer prior to exposure to the removal stimulus.

15. (canceled)

16. The method of claim 1, wherein the removal stimulus comprises one or more wavelengths in the visible to near-infrared spectrum range (about 400 nm to about 2500 nm).

17. The method of claim 3, wherein the removal stimulus comprises one or more wavelengths in the visible to near-infrared spectrum range (about 400 nm to about 2500 nm) near infrared

18. The method of claim 1, wherein the removal stimulus is produced by a device comprising one or more light sources, such as light-emitting diodes (LEDs), infrared emitters, intense pulsed light (IPL), an arc lamp, or a laser.

19. The method of claim3, wherein the removal stimulus is produced by a device comprising one or more light sources, such as light-emitting diodes (LEDs), infrared emitters, intense pulsed light (IPL), an arc lamp, or a laser.

20. (canceled)

21. A photothermal nail composition comprising:
at least one photothermal additive; and
a cosmetically acceptable carrier and/or matrix;
wherein the photothermal additive is selected from the group consisting of a carbon material, an organic dye, an inorganic compound, an inorganic element, an inorganic alloy, and combinations thereof;
wherein the at least one photothermal additive is present at a concentration ranging from about 0.001 wt. % to 99 wt. % of the total weight of the photothermal nail composition; and
wherein the at least one photothermal additive present generates heat upon exposure to a removal stimulus producing an increase in temperature in the composition of by at least about 5° C., 10° C., 15° C., 20° C., 25° C., 30° C., 35° C., 40° C., 45° C., 50° C., 60° C., 70° C., 80° C., 90° C., 100° C., 110° C., 120° C., 130° C., 140° C., 150° C., 160° C., 170° C., or 180° C. following at least about 30 seconds to about 5 minutes of exposure to the removal stimulus, as compared to the composition prior to exposure to the removal stimulus.

22. The photothermal nail composition of claim 21, wherein the cosmetically acceptable carrier and/or matrix is selected from the group consisting of a solvent, a lacquer, monomers, film-forming polymers, and combinations thereof.

23. (canceled)

24. The photothermal nail composition of claim 21, wherein the matrix is or comprises an adhesive and the photothermal nail composition is in the form of a sticker.

25. (canceled)

26. (canceled)

27. The photothermal nail composition of claim 21, wherein the carbon material is selected from the group consisting of carbon black, graphene, graphene oxide, reduced graphene oxide, single-walled carbon nanotubes, multi-walled carbon nanotubes, acetylene black, graphite, activated carbon, carbon nanofibers, and combinations thereof.

28. (canceled)

29. The photothermal nail composition of any one of claims 21-28, wherein the concentration of the photothermal additive ranges from about 0.1 wt. % to 99 wt. %, about 0.2 wt. % to 30 wt. %, about 0.5 wt. % to 20 wt. %, about 1 wt. % to 10 wt. %, or about 2 wt. % to 5 wt. % of the total weight of the photothermal nail composition.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A kit comprising the photothermal nail composition of claim 21; and
optionally one or more compositions for forming a nail coating comprising a thermally responsive material;
optionally a nail composition to form a bottom coating;
optionally one or more compositions for forming a nail product; and
optionally instructions and/or a device that is capable of producing a removal stimulus.

42. (canceled)

43. (canceled)

44. (canceled)

45. (canceled)

46. (canceled)
